(19) United States
(12) Patent Application Publication
(10) Pub. No.: US 2007/0243212 A1
(43) Pub. Date: Oct. 18, 2007

(54) Methods and compositions for vaccination of poultry

(75) Inventors: Vivian W. Doelling, Cary, NC (US); Rebecca M. Poston, Raleigh, NC (US); Cherilyn L. Heggen-Peay, Apex, NC (US); Alan P. Avakian, Raleigh, NC (US)

Correspondence Address: MYERS BIGEL SIBLEY & SAJOVEC, PO BOX 37428, RALEIGH, NC, 27627, US

(73) Assignee: Embrex, Inc.

(21) Appl. No.: 11/729,678

(22) Filed: Mar. 29, 2007

Related U.S. Application Data
(60) Provisional application No. 60/787,567, filed on Mar. 30, 2006.

(51) Int. Cl. A61K 39/295; A61K 39/08

(52) U.S. Cl. 424/201.1; 424/239.1

(57) ABSTRACT

The present invention provides methods of inducing an immune response against Clostridium species in birds, for protecting birds from Clostridium infection, and/or for protecting birds from related disorders such as necrotic enteritis. The methods can be practiced in ovo and/or post-hatch. The invention further provides compositions and methods for delivery of a composition of this invention in ovo directly to the embryo body.

### STATEMENT OF PRIORITY

The present application claims the benefit, under 35 U.S.C. § 119(e) of U.S. Provisional Application No. 60/787,567, filed Mar. 30, 2006, the entire contents of which are incorporated by reference herein.

### FIELD OF THE INVENTION

The present invention provides compositions and methods for producing an immune response in avian subjects by delivering an immunizing composition in ovo directly to the embryo of the avian subject. The present invention further provides immunogenic compositions and methods for producing an immune response to Clostridium species in avian subjects, for protecting avian subjects from Clostridium infection, and for protecting avian subjects from related disorders such as necrotic enteritis.

### BACKGROUND OF THE INVENTION

In ovo vaccination provides several advantages to the poultry industry over current control methods and potential post-hatch vaccination of birds, including the potential for uniform, automated delivery; co-administration with other vaccines in ovo, thereby reducing bird handling post-hatch; and decreased used of antibiotics.

The discovery that certain vaccines (e.g., inactivated or nonreplicating vaccines) can be delivered to the embryo body to elicit a strong immune response (similar or better to that expected when vaccinating birds at day of hatch) allows for the development of automated devices that can target the embryo body (specifically avoiding the amniotic fluid surrounding the embryo body) during in ovo application of such vaccines. In addition, knowing that inactivated vaccines should preferentially be delivered to the embryo body allows one to develop vaccine compositions that are more compatible with injection into the body of the embryo. Prior to this discovery it would not have been known or expected that inactivated vaccine would need to be administered preferentially to the embryo body to elicit a strong immune response.

Thus, the present invention is an improvement over the art by providing a more efficient way to administer inactivated (killed) vaccines in ovo. Prior to this invention there was no indication that administering inactivated vaccine to the embryonic fluids surrounding the embryo body (amniotic fluid) was any different from administering inactivated vaccine to the embryo body. The present invention demonstrates that inactivated vaccines need to be delivered in ovo to the embryo body proper rather than to the fluids surrounding the embryo body. Knowing that the embryo body is an appropriate target for optimal efficacy allows for the development of inactivated vaccines and delivery methods for the in ovo route. The invention encompasses administration of immunogenic compositions in ovo to poultry and other avian species.

Accordingly, the present invention fulfills a need in the art for improved immunogenic compositions for administration in ovo to the embryo and methods for inducing an immune response in birds, for protecting birds from infection and/or contamination by avian and other pathogens, and for protecting birds from related disorders.

Clostridium perfringens is associated with several diseases in poultry, most notably necrotic enteritis (C. perfringens type A and type C), but also including cholangiohepatitis, cellulitis, gizzard erosions, and navel infections. While these bacteria represent a component of the normal gut flora, various factors can predispose birds to disease development. Such factors include a diet having high levels of fishmeal, wheat, barley or rye; a high moisture litter; or exposure to Coccidia. Clinically, signs of disease generally include diarrhea, decreased appetite, intestinal lesions, depression, and mortality. Traditionally, these diseases are controlled by antibiotics in the feed; however, overuse of antibiotics can lead to the development of antibiotic-resistant strains of bacteria, which present a significant health risk to humans and animals. Further, in certain jurisdictions, the use of antibiotics is highly disfavored or even prohibited.

Other methods for controlling Clostridium include a diet that avoids ingredients that irritate the intestinal mucosal, e.g., a corn-soy ration; a low moisture litter having absorbent material such as wood shavings or rice hulls; the use of a competitive exclusion product to maintain a healthy balance of intestinal microflora, e.g., Primalac (Star-Labs/Forage Research, Inc., Clarksdale, Mo.), AVIGUARD™ (Bayer Corporation, Kansas City, Mo.), and BIO-MOS® (Alltech, Inc., Nicholasville, Ky.); and intense preventative water acidification or disinfection to minimize losses during a disease period. Vaccination has been used to control Clostridial diseases in other species, including cattle, sheep, goats and swine. Two main types of vaccines to C. perfringens have been developed for non-poultry species: toxoids (inactivated toxins) and bacterin-toxoids (inactivated [“killed”] bacterial cultures and inactivated toxins). Antitoxins (antibodies specific for the toxin[s]) are also used as prevention against and treatment for Clostridial diseases in non-poultry species.

As far as the inventors are aware, there are no previous reports describing in ovo administration (i.e., to an egg containing a developing avian embryo) of a C. perfringens vaccine. The use of existing toxoid or toxoid-bacterin vaccines in ovo is uncertain because such vaccines generally require the use of adjuvants, which may be harmful to the embryo, may inactivate live vaccines against other organisms that are typically administered during the in ovo period (e.g., to vaccinate against Marek's disease), and/or may be incompatible with existing in ovo injection equipment. In ovo vaccination against C. perfringens provides several advantages to the poultry industry over current control methods and potential post-hatch vaccination of birds, including the potential for uniform, automated delivery; co-administration with other vaccines in ovo, thereby reducing bird handling post-hatch; and decreased used of antibiotics.

Accordingly, there is a need in the art for improved immunogenic compositions and methods for inducing an immune response against Clostridium in birds, for protecting birds from Clostridium infection, and for protecting birds from related disorders such as necrotic enteritis.

### SUMMARY OF THE INVENTION

In some embodiments, the present invention provides a method of immunizing an avian subject against a pathogen (e.g., an avian pathogen or a non-avian pathogen carried by a bird), comprising administering in ovo during the final quarter of incubation an effective immunizing dose of a composition that induces an immune response against the pathogen, wherein the immunogenic composition is administered by in ovo injection directly into the embryo body.

In further embodiments of the invention, the composition induces an immune response to treat and/or prevent infection and/or contamination of the bird resulting from exposure to or contact with pathogens that cause the following nonlimiting examples of diseases, infections and/or disorders: coccidiosis, Marek's disease, infectious bursal disease, Newcastle disease, fowl pox infection, Clostridium spp. infection, avian influenza, infectious bronchitis, chick anemia virus infection, avian laryngotracheitis, avian metapneumovirus infection, avian reovirus infection, avian adenovirus infections, rotavirus infection, astrovirus infection, inclusion body hepatitis, egg drop syndrome, adenovirus infection, Escherichia coli infection, Mycoplasma spp. infection, Salmonella spp. infection, Campylobacter spp. infection, Listeria spp. infection, Haemophilus spp. infection, Pasteurella spp. and any combination thereof.

In yet further embodiments of the invention, the composition can comprise, consist essentially of and/or consist of a non-replicating agent that induces an immune response against an avian pathogen and/or a pathogen that causes food borne illnesses, such as Salmonella spp. infection, Campylobacter spp. infection, Listeria spp infection, Escherichia coli infection, etc. as are known in the art.

The present invention further comprises methods wherein an effective immunizing dose of two or more compositions that induce an immune response against the avian pathogen are administered in ovo to the embryo, wherein the two or more compositions are administered simultaneously or sequentially in any order.

Additionally provided herein are methods wherein an effective immunizing dose of two or more compositions that induce an immune response against the avian pathogen are administered in ovo and at least one composition is administered to the embryo, wherein the two or more compositions are administered simultaneously or sequentially in any order.

The present invention provides methods of inducing an immune response against Clostridium species (e.g., Clostridium perfringens) in birds, for protecting birds from Clostridium infection, and/or for protecting birds from related disorders such as necrotic enteritis. The methods can be practiced in ovo and/or post-hatch. The present invention further provides immunogenic compositions for inducing an immune response against Clostridium species in birds, for protecting birds from Clostridium infection, and/or for protecting birds from related disorders such as necrotic enteritis.

Accordingly, as one aspect, the present invention provides a method of immunizing an avian subject (e.g., a chicken) against necrotic enteritis comprising administering in ovo (e.g., during the final quarter of incubation) an effective immunizing dose of an immunogenic composition that induces an immune response against Clostridium perfringens, wherein the immunogenic composition is administered by in ovo injection. In certain embodiments, the immunogenic composition is administered to the amnion or to the embryo. The method can optionally be practiced in combination with other immunization regimens (e.g., vaccination against infectious bursal disease, Marek's disease, Newcastle disease and/or coccidiosis) and/or in ovo feeding of a nutrient formulation and/or enteric modulator.

As another aspect, the invention provides an immunogenic composition comprising an effective immunizing dose of an attenuated Clostridium species in a pharmaceutically acceptable carrier. In certain embodiments, the immunogenic composition further comprises an adjuvant, which can be, for example, a depot adjuvant. Representative adjuvants of this invention include but are not limited to an aluminum salt such as aluminum hydroxide gel (alum), aluminum phosphate, or algannmulin, and/or may also be a salt or mineral gels of calcium, magnesium, iron and/or zinc, and/or may be an insoluble suspension of acylated tyrosine, or acylated sugars, cationically or anionically derivatized polysaccharides, or polyphosphazenes, and/or saponins such as Quil-A, and/or oil emulsions, such as water-in-oil and water-in-oil-in-water and/or complete or incomplete Freund's or any combination thereof. In representative embodiments, the immunogenic composition comprises a water-in-oil-in-water emulsion. Optionally, the immunogenic composition can further comprise one or more additional agents that induce an immune response against other avian pathogens (e.g., an agent that induces an immune response against Eimeria, infectious bursal disease virus, Marek's disease virus and/or Newcastle disease virus) and/or a nutrient formulation and/or an enteric modulator. The one or more additional agents can be immunizing agents that produce a protective immune response against Eimeria, infectious bursal disease virus, Marek's disease virus and/or Newcastle disease virus.

As a further aspect, the invention provides an immunogenic composition comprising in a pharmaceutically acceptable carrier:

(a) an effective immunizing dose of a Clostridium toxoid, a Clostridium bacterin, a Clostridium toxin or any combination of the foregoing; and

(b) an effective immunizing dose of a coccidiosis vaccine, a Marek's disease vaccine, an infectious bursal disease vaccine, a Newcastle disease vaccine, a fowl pox vaccine, or any combination of the foregoing.

The invention also provides an immunogenic composition comprising in a pharmaceutically acceptable carrier:

(a) an effective immunizing dose of a Clostridium toxoid, a Clostridium bacterin, a Clostridium toxin or any combination of the foregoing; and

(b) an oil emulsion.

As still another aspect, the invention provides an immunogenic composition comprising in a pharmaceutically acceptable carrier:

(a) an effective immunizing dose of a Clostridium toxoid, a Clostridium bacterin, a Clostridium toxin or any combination of the foregoing; and

(b) an adjuvant comprising an aluminum derived adjuvant, a saponin, an oil, or any combination of the foregoing.

In further embodiments, the present invention provides a method of immunizing an avian subject against infection by a Clostridium species, comprising administering to the avian subject an effective immunizing dose of a Clostridium bacterin-toxoid composition by in ovo injection during the final quarter of incubation. In some embodiments of the invention, the species of Clostridium can be Clostridium perfringens.

The present invention also provides a method of immunizing an avian subject against infection by a Clostridium species, comprising administering to the avian subject an effective immunizing dose of a recombinant toxin or immunogenic fragment thereof of a Clostridium species by in ovo injection during the final quarter of incubation. In some embodiments, the toxin or immunogenic fragment thereof is a Clostridium perfringens toxin or immunogenic fragment thereof.

These and other aspects of the invention are set forth in more detail in the description of the invention below.

### DETAILED DESCRIPTION OF THE INVENTION

Certain aspects of the present invention are based on the unexpected discovery that certain antigenic or immunogenic compositions allow for a more effective immune response in birds when the composition is administered in ovo directly to the embryo body

Thus, in one embodiment, the present invention provides a method of immunizing an avian subject against a pathogen, which can be an avian pathogen and/or a non avian pathogen carried by an avian subject (e.g., human food borne pathogen), comprising administering in ovo during the final quarter of incubation an effective immunizing dose of a composition that induces an immune response against the avian pathogen, wherein the immunogenic composition is administered by in ovo injection directly into the embryo body. In the methods of this invention, the composition can be administered directly into skeletal muscle tissue in the embryo and the skeletal muscle tissue can be, but is not limited to, breast muscle tissue and pipping muscle tissue. In further embodiments, the composition can be administered directly into the embryo into the head, neck, shoulder, wing, back, breast, leg or any combination thereof.

Further, in the methods of this invention, the composition can be administered subcutaneous in the embryo body. In further embodiments, the composition can be administered subcutaneous into the head, neck, shoulder, wing, back, breast, leg or any combination thereof.

Any suitable route of administration into the embryo is suitable in employing the methods of this invention. For example, the composition can be administered to the embryo subcutaneously, intra-dermally, intravenously, intramuscularly, intra-abdominally or any combination thereof.

The avian subject of this invention can be any avian and in certain embodiments, the subject can be a chicken, turkey, duck, goose, pheasant, quail, partridge, guinea, ostrich, emu or peafowl, as well as any other commercially processed avian and/or any avian, the eggs of which are accessible for handling in the methods of this invention

In embodiments wherein the subject is a chicken, it is desirable to administer the composition of this invention in ovo during the period from day 15 through day 20 of incubation, and in particular embodiments, the composition can be administered on day 18 or day 19 of incubation. When the subject is a turkey, the composition of this invention can be administered during the period from day 21 through day 28 of incubation and in particular embodiments, the compositions can be administered on day 24 or day 25 of incubation. In other embodiments wherein the subject is a goose, the composition of this invention can be administered during the period from day 23 through day 31 of incubation and in particular embodiments, the compositions can be administered on day 28 or day 29 of incubation. In further embodiments wherein the subject is a duck, the composition of this invention can be administered during the period from day 21 through day 28 of incubation and in particular embodiments, the compositions can be administered on day 25 or day 26 of incubation.

For other avian species, the final quarter of incubation and thus the optimal range of days for in ovo administration of a composition of this invention can be determined according to methods well known in the art. For example, a muscovy duck has an incubation period in the range of 33-35 days, a ringneck pheasant has an incubation period of 23-24 days, a Japanese quail has an incubation period of 17-18 days, a bobwhite quail has an incubation period of 23 days, a chuckar partridge has an incubation period of 22-23 days, a guinea has an incubation period of 26-28 days and a peafowl has an incubation period of 28 days.

In particular embodiments of this invention, the composition can comprise, consist essentially of and/or consist of an immunogenic composition and an adjuvant. Nonlimiting examples of adjuvants of this invention include an aluminum derived adjuvant, a saponin, mineral gels, polyanions, pluronic polyols, saponin derivatives, lysolecithin and other similar surface active substances, glycosides, all types of oils and any combination thereof. In particular embodiments of this invention, the composition can comprise a water-in-oil-in-water emulsion.

As contemplated herein, in some embodiments of the present invention, the composition of this invention can comprise an adjuvant, which in particular embodiments, can be an adjuvant such as an aluminum derived adjuvant (e.g., aluminum hydroxide), a saponin (e.g., Quil-A including QuilA QS21), or an oil (such as Complete or Incomplete Freund's adjuvant), in any combination.

Further nonlimiting examples of an adjuvant of this invention include mineral salts (e.g., aluminum hydroxide; aluminum phosphate; calcium phosphate), oil emulsions and surfactant based formulations (e.g., Freund's emulsified oil adjuvants; Arlacel A; mineral oil; emulsified peanut oil adjuvant (adjuvant 65); MF59 (microfluidized detergent stabilized oil-in-water emulsion); QS21 (purified saponin); AS02 [SBAS2] (oil-in-water+MPL+QS21); Montanide ISA-51; ISA-720 (stabilized water-in-oil emulsion), bacterial products and derivatives [e.g., Bordatella pertussis; Corynebacterium granulosum derived P40 component; lipopolysaccharide (adjuvant for both humoral and cell-mediated immunity); Mycobacterium and its components (MDPs, not acceptable adjuvants in humans); cholera toxin (mucosal adjuvant)], microbial derivatives (natural and synthetic) [e.g., monophosphoryl lipid A (MPL); Detox (MPL+M. phlei cell wall skeleton); AGP [RC-529] (synthetic acylated monosaccharide); DC-Chol (lipoidal immunostimulators able to self-organize into liposomes); OM-174 (lipid A derivative); CpG motifs (synthetic oligonucleotides containing immunostimulatory CpG motifs); modified LT and CT (genetically modified bacterial toxins to provide non-toxic adjuvant effects)], endogenous chicken immunomodulators [cytokines; antibodies; hGM-CSF or hIL-12 (cytokines that can be administered either as protein or plasmid encoded); Immudaptin (C3d tandem array); Squalene], particulate adjuvants [virosomes (unilamellar liposomal vehicles incorporating antigen); AS04 ([SBAS4] Al salt with MPL); ISCOMs (structured complex of saponins and lipids); polylactide co-glycolide (PLG), and inert vehicles (gold particles; silver particles).

Additional adjuvants of this invention can include mineral gels, polyanions, pluronic polyols, saponin derivatives, lysolecithin and other similar surface active substances. Further adjuvants can include toll-like receptor (TLR) agonists, including, for example, agonists of TLR-1 (e.g. tri-acyl lipopeptides); agonists of TLR-2 [e.g. peptidoglycan of gram-positive bacteria like streptococci and staphylococci; lipoteichoic acid]; agonists of TLR-3 (e.g. double-stranded RNA and their analogs such as poly 1:C); agonists of TLR-4 [e.g. lipopolysaccharide (endotoxin) of gram-negative bacteria like Salmonella and E. coli]; agonists of TLR-5 (e.g. flagellin of motile bacteria like Listeria); agonists of TLR-6 [e.g. with TLR-2 peptidoglycan and certain lipids (diacyl lipopeptides)]; agonists of TLR-7 [e.g. single-stranded RNA (ssRNA) genomes of such viruses as influenza, measles, and mumps; and small synthetic guanosine-base antiviral molecules like loxoribine and ssRNA and their analogs]; agonists of TLR-8 (e.g. binds ssRNA); agonists of TLR-9 (e.g. unmethylated CpG of the DNA of the pathogen and their analogs; agonists of TLR-10 (function not defined) and TLR-11—(e.g. binds proteins expressed by several infectious protozoans (Apicomplexa). Chickens have a well developed TLR system with approximately 10 TLRs broadly similar to those detected in mammals.

More examples of adjuvants of this invention include complement receptors (secreted PRRs), wherein C3d (complement component is activated by microbial CHO. The complement pathway leads to opsonization of the pathogen and quick phagocytosis.

In further embodiments, an adjuvant of this invention can be an amino acid sequence that is a peptide, a protein fragment or a whole protein that functions as the adjuvant, or the adjuvant can be a nucleic acid encoding a peptide, protein fragment or whole protein that functions as an adjuvant. As used herein, “adjuvant” describes a substance, which can be any immunomodulating substance capable of being combined with the polypeptide or nucleic acid vaccine to enhance, improve or otherwise modulate an immune response in a subject without deleterious effect on the subject.

An adjuvant of this invention can be, but is not limited to, for example, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153 (the entire contents of which are incorporated herein by reference), or a less reactogenic composition where the QS21 is quenched with cholesterol as disclosed in PCT publication number WO 96/33739 (the entire contents of which are incorporated herein by reference). A particularly potent adjuvant formulation involving QS21 3D-MPL & tocopherol in an oil in water emulsion is described in PCT publication number WO 95/17210 (the entire contents of which are incorporated herein by reference).

The compositions and methods of the present invention can be employed to induce an immune response to treat and/or prevent such diseases and disorders as coccidiosis, Marek's disease, infectious bursal disease, Newcastle disease, fowl pox infection, Clostridium spp. infection (e.g., necrotic enteritis, gangrenous dermatitis, cholangiohepatitis, cellulites, ulcerative enteritis, botulism, Tyzzer's disease), avian influenza, infectious bronchitis, chick anemia virus infection, avian laryngotracheitis, avian metapneumovirus, avian reovirus infection, avian adenovirus infections, rotavirus infection, astrovirus infection, inclusion body hepatitis, egg drop syndrome, adenovirus infection, Escherichia coli infection, Mycoplasma spp. infection, Salmonella spp. infection, Campylobacter spp. infection, Listeria spp., Haemophilus spp. infection, Pasteurella spp.; Bordetella spp., Staphylococcus spp., Streptococcus spp., Mycobacterium spp., Erysipelothrix spp. and any combination thereof.

Thus, in certain embodiments, the composition of this invention can comprise, consist essentially of and/or consist of an antigen or immunogen from Marek's disease virus, infectious bronchitis virus, Mycoplasma spp., avian leucosis virus, reovirus, poxvirus, adenovirus, cryptosporidium, chicken infectious anemia virus, Pasteurella species, avian influenza virus, Newcastle disease virus (NDV), infectious bursal disease virus (IBDV), Rous sarcoma virus, Escherichia coli, Eimeria species such as Eimeria tenella (causing coccidiosis), Haemophilus species, Mycoplasma, Listeria species, Salmonella species, Campylobacter species, Clostridium species (e.g., C. perfringens, C. septicum, C. sordellii, C. difficile, C. novyi, C. botulinum, C. colinum, C. chauvoei, C. fallax, C. sporogenes, and C. piliforme) and any combination thereof.

The present invention is intended to encompass methods and compositions to immunize avians against pathogens, which can be pathogens that cause disease in avians and/or pathogens that are carried by avians (contaminated avians) and are passed on to humans and other animals who handle or eat such contaminated avians. Thus, the present invention provides compositions comprising, consisting essentially of and/or consisting of a non-replicating agent that induces an immune response against an avian pathogen or against a pathogen that causes disease in other animals by contact with or ingestion of eggs or meat or other body parts of a contaminated avian. Such pathogens can include but are not limited to Salmonella spp., Campylobacter spp., Listeria, Escherichia coli, Erysipelothrix spp., Mycobacterium spp., Clostridium spp., etc.

In some embodiments, the methods of the invention described herein, can further comprise the step of administering a booster dose of the composition of this invention to the avian subject post hatch.

In further embodiments of the methods of this invention, an effective immunizing dose of two or more compositions that induce an immune response against the avian pathogen are administered in ovo to the embryo, wherein the two or more compositions are administered simultaneously or sequentially in any order. Thus, the compositions and methods of this invention can be employed using apparatus and technology that comprises administering multiple compositions at a single site, multiple compositions at multiple sites and/or a single composition at multiple sites. Such methods can employ a single entry site into the egg or multiple entry sites into the egg. Nonlimiting examples of such apparatus and technology are described in U.S. Pat. No. 4,903,635, U.S. Pat. No. 5,136,979, U.S. Pat. No. RE 35,973, U.S. Pat. No. 5,339,766, U.S. Pat. No. 6,032,612, U.S. Pat. No. 6,286,455, U.S. Pat. No. 5,158,038, U.S. Pat. No. 6,601,534 and U.S. Pat. No. 6,981,470, the entire contents of each of which are incorporated by reference herein.

Also included herein are methods wherein an effective immunizing dose of two or more compositions that induce an immune response against the avian pathogen are administered in ovo and at least one composition is administered to the embryo, wherein the two or more compositions are administered simultaneously or sequentially in any order. In some embodiments of these methods, at least one composition can be administered to the amnion, which includes the amniotic fluid, embryo body and yolk sac and/or the at least one composition can be administered directly into the amniotic fluid, the embryo body and/or the yolk sac, individually or in any combination.

In some embodiments of this invention, the methods can further comprise administering in ovo an immune stimulant at any time during incubation, wherein the immune stimulant and the composition are administered simultaneously or sequentially in any order. The methods of this invention can also further comprise administering in ovo a nutrient formulation, an enteric modulator, or a combination thereof at any time during incubation, wherein the nutrient formulation, the enteric modulator or a combination thereof and the composition are administered simultaneously or sequentially in any order.

There are several aspects of avian embryonic development that make the embryo an attractive target for immunization. First, since the greatest period of embryonic development occurs in the egg outside the maternal reproductive tract, the embryo can be easily accessed for the introduction of compositions such as the immunogenic compositions of this invention.

Second, the fact that the egg is a multi-compartmentalized unit can be exploited to deliver biological materials to specific embryonic sites. For example, the yolk sac in the early embryo functions to manufacture blood. Immediately prior to hatching, the yolk sac serves a primarily nutritional function and in part is taken into the intestinal tract and thereby transported to the cecal pouches during and after hatch. Therefore, yolk sac administration of materials can lead to both embryonic cecal or vascular system delivery. In addition, administration of a composition of this invention can be efficiently carried out by injection onto the chorio-allantoic membrane or onto the air cell membrane. Finally, access to the embryonic musculature compartment can be achieved by direct embryonic injection at transfer in the last quarter of incubation, and in chickens this is generally carried out from day 17 through day 19 of incubation.

The immunogenic composition may be introduced into any region of the egg, including the air cell, the albumen, the chorio-allantoic membrane, the yolk sac, the yolk, the allantois, the amnion, or directly into the embryonic bird. In a particular embodiment of the invention, the composition is introduced into muscle tissue of the embryonic bird, and in a other embodiments, the composition is introduced into skeletal muscle tissue. In certain embodiments, introduction of a nucleic acid molecule encoding a protein which remains within the muscle cell can be used to administer a immunogenic protein directly and specifically to muscle cells. Alternatively, a nucleic acid molecule can be introduced which encodes a protein which will be secreted from the muscle cell and this method can be used to deliver a protein to the entire body of the bird through contact between the muscle tissue and plasma. Exemplary skeletal muscle tissue introduction sites are breast muscle and pipping muscle tissue, which are located near the eggshell and thus are relatively easily reached by injection apparatus without damage to other embryonic structures.

Any suitable means may be used for introducing the composition of this invention in ovo, including in ovo injection, high pressure spray through the egg shell, and ballistic bombardment of the egg with microparticles carrying the composition. In some embodiments, the composition is administered by depositing an aqueous, pharmaceutically acceptable solution in the muscle, which solution contains the composition to be deposited.

Where in ovo injection is used, the mechanism of injection is not critical, but it is preferred that the method not unduly damage the tissues and organs of the embryo or the extraembryonic membranes surrounding it so that the treatment will not decrease hatch rate. Preferred injection sites are intra-muscular and subcutaneous. Preferred muscle tissue injection sites are skeletal muscle, and more particularly breast muscle and pipping muscle tissue, which are located near the eggshell and thus are relatively easily reached by injection apparatus without damage to other embryonic structures and without compromising the protection afforded by the eggshell. A syringe fitted with a needle of about 18 to 26 gauge is suitable for the purpose. Depending on the precise stage of development and position of the embryo, a ¾ to 4 inch needle will terminate either in the fluid above the chick or in the chick itself. A pilot hole may be punched or drilled through the shell prior to insertion of the needle to prevent damaging or dulling of the needle. If desired, the egg can be sealed with a substantially bacteria-impermeable sealing material such as wax or the like to prevent subsequent entry of undesirable bacteria.

In various embodiments of this invention, the composition is administered to the embryo body in a needle having a length from about ¾ inch to about 4 inches (e.g., 1 inch, 1.25 inch, 1.5 inch, 1.75 inch, 2.0 inch, 2.25 inch, 2.5 inch, 2.75 inch, 3.0 inch, 3.25 inch, 3.5 inch, 3.75 inch, or 4.0 inch). Furthermore, a needle of this invention can have a gauge ranging from 15 g to 28 g (e.g., 15 g, 16 g, 17 g, 18 g, 19 g, 20 g, 21 g, 22 g, 23 g, 24 g, 25 g, 26 g, 27 g or 28 g). In some embodiments, the needle can have a blunt end and in some embodiments, the needle can have a beveled end with a bevel angle of about 10° to about 45° (e.g., 11°, 12°, 13°, 14°, 15°, 16°, 17°, 18°, 19°, 20°, 21°, 22°, 23°, 24°, 25°, 26°, 27°, 28°, 29°, 30°, 31°, 32°, 33°, 34°, 35°, 36°, 37°, 38°, 39°, 40°, 41°, 42°, 43°, 44°, or 45°).

In particular embodiments of this invention, in the methods of administering a composition of this invention in ovo, the needle can pass through the shell at the large end of an egg at an angle offset by about 1° to about 20° (e.g., 1°, 2°, 3°, 4°, 5°, 6°, 7°, 8°, 9°, 10°, 11°, 12°, 13°, 14°, 15°, 16°, 17°, 18°, 19°, or 20°) from the long axis of the egg.

The present invention also provides methods wherein the composition of this invention is administered in ovo with an automated injection device.

It is envisioned that a high speed automated injection system for avian embryos will be particularly suitable for practicing the present invention. Numerous such devices are available, exemplary being the EMBREX INOVOJECT® system (described in U.S. Pat. No. 4,681,063 to Hebrank), and U.S. Pat. Nos. 4,040,388, 4,469,047, and 4,593,646 to Miller. The disclosure of these references and all references cited herein are to be incorporated herein by reference. All such devices, as adapted for practicing the present invention, comprise an injector containing the DNA as described herein, with the injector positioned to inject an egg carried by the apparatus with the DNA. In addition, a sealing apparatus operatively associated with the injection apparatus may be provided for sealing the hole in the egg after injection thereof.

The currently preferred apparatus for practicing the present invention is disclosed in U.S. Pat. No. 4,681,063 to Hebrank and U.S. Pat. No. 4,903,625 to Hebrank, the disclosures of which are incorporated herein by reference. This device comprises an injection apparatus for delivering fluid substances into a plurality of eggs and suction apparatus which simultaneously engages and lifts a plurality of individual eggs from their upwardly facing portions and cooperates with the injector for injecting the eggs while the eggs are engaged by the suction apparatus. The features of this apparatus may be combined with the features of the apparatus described above for practicing the present invention. Those skilled in the art will appreciate that this device can be adapted for injection into any portion of the egg by adjusting the penetration depth of the injector, as discussed in greater detail below.

Embodiments of an injection method and apparatus that can be employed in the present invention are described in U.S. Pat. No. 6,032,612 (multisite in ovo injection apparatus), U.S. Pat. No. 6,244,214 (apparatus for in ovo injection and detection of information regarding the interior of the egg), U.S. Pat. Nos. 6,176,199, 6,510,811 and 6,834,615 (methods of localizing allantoic fluid in an egg), U.S. Pat. No. 7,089,879 (methods for manipulating air cells within avian eggs) and U.S. Pat. No. 7,165,507 (methods and apparatus for accurately positioning a device within the subgerminal cavity of an egg), the entire contents of each of which are incorporated by reference herein.

Thus, in some embodiments, the method and apparatus is essentially as described in one or more of the patents listed above, and involves positioning an elongate injector or injection needle at the large end of the egg at an angle (A) offset from the long axis of said egg, the angle selected so that the needle is directed toward the shoulder or breast of said embryo. The needle is then inserted through the shell of the egg, along an essentially linear path of travel, to a depth sufficient to pass into the shoulder or breast of the embryo. The substance to be deposited in the egg, which may be either a liquid or a syringable (e.g., injectable) solid (but is generally an aqueous solution containing the composition of this invention as described herein), is then injected through the needle. In some embodiments, the needle is withdrawn along the essentially linear path of travel, and the step of injecting the substance is carried out concurrently with the step of withdrawing the needle so that the substance is administered along the path of travel within the egg. The angle of offset (A) is sufficient to enhance the probability of injecting into shoulder or breast muscle. Typically, the angle is 1 to 20 degrees, and preferably the angle is from 2 to 3 degrees. As one example, the needle may be inserted to a depth sufficient beneath the egg shell to pass into or pass into and through the shoulder or breast of the embryo. The apparatus may be modified to include means operably associated with the apparatus for positioning the egg at an angle with respect to the needle to achieve said angle (A), such as by mounting and positioning the needle(s) at an angle with respect to the suction apparatus.

In a particular example, the methods of the present invention can be practiced with the apparatus described in U.S. Pat. No. 6,244,214 to Hebrank (the entire contents of which are incorporated by reference herein), wherein an apparatus (e.g., a “smart probe”) for identifying the specific structure and or compartment within an egg that is in contact with a needle that has penetrated the shell of the egg, and methods for employing the apparatus for delivering compositions into specific structures and/or compartments within an egg are described.

Thus, in certain embodiments, the present invention provides a method of introducing a substance into the muscle of a chicken in ovo, comprising: a) obtaining a chicken egg, wherein said egg contains a chicken embryo in its last quarter of incubation prior to hatch; b) positioning an elongate injection needle at the large end of the egg at an angle offset about 1 to 5 degrees from the long axis of said egg, said angle selected so that the needle is directed toward the shoulder or breast of said embryo; c) inserting said needle through the shell of said egg along an essentially linear path of travel to a depth of about ⅞ inch to 1.5 inch into the shoulder or breast of said embryo; and d) injecting said substance into the egg through said needle.

In yet other embodiments, a method is provided herein for introducing a substance into the muscle of a chicken in ovo, comprising: a) obtaining a chicken egg, wherein said egg contains a 17-19 day chicken embryo; b) positioning an elongate injection needle at the large end of the egg at an angle offset about 1 to 5 degrees from the long axis of said egg, said angle selected so that the needle is directed toward the shoulder or breast of said embryo; c) inserting said needle through the shell of said egg along an essentially linear path of travel to a depth of about ⅞ inch to 1.5 inch into the shoulder or breast of said embryo; and d) injecting said substance into the egg through said needle. In such methods, the needle can be inserted to a depth sufficient to pass into and through the shoulder or breast of said embryo.

Also provided herein is an apparatus for simultaneously injecting muscle tissue of chicken embryos in a plurality of eggs during days 17 to 19 of incubation, said device comprising: engaging means for engaging said plurality of eggs; injection means cooperating with said engaging means for inserting an elongate needle through the shells of said eggs along an essentially linear path of travel to a depth of about ⅞ inch to 1.5 inch into the shoulder or breast of said embryo; and positioning means for positioning said elongate injection needle at the large end of said egg at an angle of about 1 to 5 degrees of offset from the long axis of said egg so that said needle is directed toward the shoulder or breast of said embryo. In such an apparatus, the engaging means can comprise suction means for simultaneously lifting a plurality of individual eggs.

In further embodiments of this invention, compositions and methods are provided to induce an immune response to Clostridium species in an avian subject. As one example, the acute enterotoxemia called necrotic enteritis in birds is caused by Clostridium perfringens (types A and C have been associated with the avian disease). Necrotic enteritis can occur when Clostridia-contaminated feed and litter are ingested by birds, and the organism grows in the intestine and then forms spores. This sporulation process causes the release of the alpha and beta toxins responsible for intestinal necrosis (particularly in the jejunum and ileum). Clinical signs include depression, decreased appetite and diarrhea. Acute mortality can occur. Predisposing factors for C. perfringens infection and necrotic enteritis include diet and damage to the intestinal mucosa. In the commercial poultry context, the majority of cases of necrotic enteritis occur in broiler chickens from two to five weeks of age. Thus, particular embodiments of the present invention are directed to immunogenic compositions and methods that protect birds against Clostridium perfringens and necrotic enteritis, for example, by reducing infection rates and/or by reducing the severity of the infection and/or disease.

The present invention will now be described with reference to particular embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Further, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

The terms “avian” and “avian subjects” or “bird” and “bird subjects” as used herein, are intended to include males and females of any avian or bird species, and in particular are intended to encompass poultry which are commercially raised for eggs, meat or as pets. Accordingly, the terms “avian” and “avian subject” or “bird” and “bird subject” encompass chickens, turkeys, ducks, geese, quail, pheasant, parakeets, parrots, cockatoos, cockatiels, ostriches, emus and the like. In particular embodiments, the subject is a chicken or a turkey. Commercial poultry includes broilers and layers, which are raised for meat and egg production, respectively.

In particular embodiments, the subject is one that is at risk for or is susceptible to infection or disease caused by Clostridium species (e.g., necrotic enteritis caused by infection with Clostridium perfringens). Risk factors for necrotic enteritis are known in the art and include, but are not limited to, dietary factors (e.g., a diet high in wheat, barley, rye or fishmeal), poor litter conditions, and/or exposure to Eimeria (e.g., natural exposure or live Eimeria vaccines). Thus, in particular embodiments, the inventive compositions and methods can advantageously be employed to reduce the severity and/or incidence of necrotic enteritis among birds that have been vaccinated against coccidiosis or in flocks that are experiencing an outbreak of coccidiosis.

Other diseases and disorders caused by infection of avians with Clostridium include but are not limited to necrotic enteritis, gangrenous dermatitis, cholangiohepatitis, cellulites, ulcerative enteritis, botulism and Tyzzer's disease. Thus, the present invention provides immunogenic compositions and methods of their use to protect avians against infection by, for example, Clostridium perfringens, C. septicum, C. sordellii, C. difficile, C. novyi, C. botulinum, C. colinum, C. chauvoei, C. fallax, C. sporogenes and/or C. piliforme.

The avian subject of this invention can be a live, embryonic bird in ovo or may be a hatched bird, including newly-hatched (i.e., about the first one, two or three days after hatch), adolescent, and adult birds.

In particular embodiments, the bird is about six-, five-, four-, three-, two- or one-week of age or less. In other representative embodiments, the avian subject is a naïve subject, i.e., has not previously been exposed to the antigen against which immunity is desired.

The term “administering in ovo” or “in ovo administration,” as used herein, unless otherwise indicated, means administering an immunogenic composition (e.g., a vaccine) to a bird egg containing a live, developing embryo by any means of penetrating the shell of the egg and introducing the immunogenic composition. Such means of administration include, but are not limited to, in ovo injection of the immunogenic composition.

The present invention provides methods of administering an immunogenic composition to a subject to induce an immune response against Clostridium species, optionally a protective immune response, in the subject. The immunogenic composition can be administered to any suitable compartment of the egg (e.g., allantois, yolk sac, amnion, air cell and/or into the avian embryo itself), as would be apparent to one skilled in the art. Methods of administration into the embryo include without limitation parenteral administration, such as for example, subcutaneous, intramuscular, intra-abdominal, intravenous, and/or intra-articular administration. In particular embodiments, the immunogenic composition is administered to the amnion (e.g., by injection axially through the large end of the egg).

The immunogenic composition can be administered to the egg by any suitable method. In particular embodiments, the immunogenic composition is administered via injection. The mechanism of egg injection is not critical, but generally should be selected so that the method does not damage the tissues and organs of the embryo or the extraembryonic membranes surrounding it to such an extent that the treatment unduly decreases hatch rate. A syringe fitted with a needle of about 18 to 23 gauge is generally suitable for the purpose. Examples of needles suitable for this invention include needles having the following gauges: 18, 19, 20, 21, 22, or 23 gauge. A needle of this invention can be at least ½ inch, ⅚ inch, ¾ inch, ⅞ inch, 1 inch, 1 and ¼ inch, 1 and ⅜ inch, 1 and ½ inch, 1 and ⅝ inch, 1 and ¾ inch, 1 and ⅞ inch or 2.0 inches in length. Examples of a bevel range of a needle of this invention is from about 5 degrees to about 45 degrees (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 45, 56, 37, 38, 39, 40, 41, 42, 43, 44 or 45 degrees) In certain embodiments of this invention the bevel range of a needle of this invention is from about 12 degrees to 20 degrees. A pilot hole can be punched or drilled through the shell prior to insertion of the needle to prevent damaging or dulling of the needle. If desired, the egg can be sealed with a substantially bacteria-impermeable sealing material such as wax or the like to prevent subsequent entry of undesirable bacteria.

A high-speed automated egg injection system for avian embryos is particularly suitable for practicing the present invention. Numerous such devices are available, exemplary being those disclosed in U.S. Pat. Nos. 4,681,063 and 4,903,635 to Hebrank and U.S. Pat. Nos. 4,040,388, 4,469,047, and 4,593,646 to Miller, the entire contents of each of which are incorporated herein. Such devices, as adapted for practicing the present invention, generally comprise an injector containing the immunogenic composition, with the injector positioned to inject an egg carried by the apparatus with the immunogenic composition. In addition, if desired, a sealing apparatus operatively associated with the injection apparatus may be provided for sealing the hole in the egg after injection thereof.

In one embodiment, the apparatus for practicing the present invention can be as disclosed in U.S. Pat. No. 4,681,063 to Hebrank and U.S. Pat. No. 4,903,625 to Hebrank, the disclosure of which are incorporated herein by reference. This device comprises an injection apparatus for delivering fluid substances into a plurality of eggs and suction apparatus which simultaneously engages and lifts a plurality of individual eggs from their upwardly facing portions and cooperates with the injector for injecting the eggs while the eggs are engaged by the suction apparatus. Those skilled in the art will appreciate that this device can be adapted for injection into any portion of the egg by adjusting the penetration depth of the injector, as is known in the art. The present invention can also be practiced with the apparatus and methods described in U.S. Pat. No. 6,244,214 to Hebrank (the entire contents of which are incorporated by reference herein), wherein an apparatus for identifying the specific structure and or compartment within an egg that is in contact with a needle that has penetrated the shell of the egg and methods for employing the apparatus for delivering compositions into specific structures and/or compartments within an egg are described.

The appropriate volume of the immunogenic composition to be administered will depend on the size of the egg being treated, with ostrich eggs obviously being capable of taking more volume than chicken eggs. In particular embodiments, the immunogenic composition is administered in a volume from about 10 to about 500, 1000 or 2000 μl or more, including any number between 10 and 2000, even if not explicitly recited herein, with exemplary volumes including 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 626, 650, 675, 700, 725, 752, 775, 800, 850, 900, 1000, 1100, 1200, 1300, 1400, 1500, 1600, 1700, 1800, 1900 or 2000 μl. Other suitable volumes for delivering the immunogenic composition can be readily determined by those skilled in the art.

According to particular embodiments of the present invention, the eggs (i.e., embryonic birds) administered the immunogenic composition are in the last half or the last quarter of in ovo incubation (i.e., of embryonic development). For example, for chicken eggs the last half of incubation is from about the twelfth to twentieth day of incubation (e.g., E12, E13, E14, E15, E16, E17, E17.5, E18, E18.5, E19, E19.5 and/or E20), and the last quarter of in ovo incubation is from about the fifteenth to twentieth day of incubation (e.g., E15, E15.5, E16, E16.5, E17, E17.5, E18, E18.5, E19 E19.5 and/or E20). In particular embodiments, the egg is administered the immunogenic composition on about the eighteenth (E18 or E18.5) or nineteenth (E19 or E19.5) day of in ovo incubation. In other embodiments, turkey eggs are administered the immunogenic composition on about the fourteenth to twenty-seventh day of incubation (E14, E15, E16, E17, E18, E19, E20, E21, E21.5, E22, E22.5, E23, E23.5, E24, E24.5, E25, E25.5, E26 and/or E27), on about the twenty-first to twenty-seventh day of incubation (e.g., E21, E21.5, E22, E22.5, E23, E23.5, E24, E24.5, E25, E25.2, E26, E26.5 and/or E27), or on about the twenty-fifth (E25 or E25.5) day of incubation. Those skilled in the art will appreciate that the present invention can be carried out at any predetermined time in ovo, as long as the administration results in a desired immune response to the immunogenic composition without undue levels of morbidity and/or mortality among the treated subjects.

Alternatively or additionally, the invention can be practiced to administer an immunogenic composition to a hatched bird, including newly hatched (i.e., about the first one, two and/or three days after hatch), adolescent, and/or adult birds. In certain embodiments, administration is within about the first six, five, four, three and/or two weeks after hatch and/or even within about the first week after hatch. According to one aspect of the invention, administration is within the first three weeks after hatch. In other embodiments, the immunogenic composition is administered in ovo (e.g., in the last quarter of in ovo incubation) and a booster is administered post-hatch (e.g., within about the first one, two or three days or one, two or three weeks post-hatch).

The methods of the invention can be distinguished from maternal vaccination approaches in which older female birds (e.g., hens at about 10-15 weeks of age) are vaccinated for the purpose of providing passive immunity to their off-spring. Such birds are probably not naïve, i.e., they have already been exposed to Clostridium (e.g., Clostridium perfringens) and are not being immunized for the purpose of protecting the vaccinated bird but instead to protect the offspring by passive transfer of antibodies.

The immunogenic compositions of the present invention can be administered to hatched birds by any suitable means. Exemplary means are oral administration (e.g., in the feed or drinking water), intramuscular injection, subcutaneous injection, intravenous injection, intra-abdominal injection, eye drop and/or nasal spray. Further, birds can be administered immunogenic compositions in a spray cabinet, i.e., a cabinet in which the birds are placed and exposed to a mist containing vaccine, or by course spray.

The invention can be practiced to protect a bird from necrotic enteritis. By “protect,” “protecting,” and “protection” and like terms it is meant any level of protection from necrotic enteritis which is of some benefit in a population of subjects, such that there is a reduction in the incidence and/or the severity of the disease among treated birds whether in the form of decreased mortality, decreased lesions, improved body weight, improved feed conversion ratios and/or the reduction of any other detrimental effect of the disease, regardless of whether the protection is partial or complete. Those skilled in the art will understand that protection can be assessed from a plurality of treated birds as compared with untreated birds, even if individual treated birds are not protected. In particular embodiments, the invention provides a method of reducing the incidence of necrotic enteritis among a plurality of birds that are administered the immunogenic compositions of the invention. Also provided is a method of reducing morbidity and/or mortality among a plurality of birds that are treated according to the present invention.

By “prime,” “primed” or “priming” (and grammatical variations thereof) as used herein, it is meant to initiate an active immune response that is less than the protective until a second dose (booster) has given at a later time post hatch.

By “reduce,” “reduced,” “reducing,” and “reduction” (and grammatical variations thereof), as used herein, it is meant a decrease in the indicated infection- or disease-related parameter (e.g., infection, morbidity, mortality, incidence of necrotic enteritis, lesions and the like) that is of some value or benefit to the user (e.g., commercial value), for example, a decrease of at least about 20%, 25%, 35%, 50%, 75%, 80%, 85%, 90%, 95%, 97% or more as compared with untreated birds.

The invention also provides methods of protecting birds from infection by Clostridium species, which results in any level of protection that is of some benefit in a population of subjects, such that there is a reduction in the incidence and/or the severity of Clostridium infection among treated birds.

The invention can also be practiced to induce an immune response to Clostridium. As used herein, the term “induce (or grammatical variations thereof) an immune response against Clostridium” is intended to encompass agents that induce an immune response against the organism itself and/or the toxins produced by the organism, by means of passive transfer or active immune response. Optionally, the immune response that is induced is a protective immune response, for example, in vaccination methods. Protection is not required if there is some other purpose for inducing the immune response, for example, for research purposes or to produce antibody for passive immunizations or as a reagent (e.g., to detect, isolate and/or identify Clostridium species).

As used herein, unless indicated otherwise, “C. perfringens” is intended to include C. perfringens type A and/or C. perfringens type C and/or any other C. perfringens type that is implicated in the etiology of necrotic enteritis in birds. In particular embodiments, the invention provides methods of protecting birds from infection by C. perfringens type A and/or C. perfringens type C. The invention also provides methods of inducing an immune response against C. perfringens type A and/or C. perfringens type C. Different types of C. perfringens and strains thereof are well-known in the art. See, e.g., AMERICAN ASSOCIATION OF AVIAN PATHOLOGISTS, A LABORATORY MANUAL FOR THE ISOLATION AND IDENTIFICATION OF PATHOGENS (3d. ed. 1989).

The term “effective immunizing dose,” as used herein, unless otherwise indicated, means a dose of the immunogenic composition sufficient to induce a protective immune response in the treated birds that is greater than the inherent immunity of non-immunized birds. In the case of birds treated in ovo, an “effective immunizing dose” indicates a dose sufficient to induce a protective immune response in the hatched birds that have been treated in ovo that is greater than the inherent immunity of birds that were not immunized in ovo. An effective immunizing dose in any particular context can be routinely determined using methods known in the art.

An “effective immunizing dose” can comprise one or more (e.g., two or three) doses of the immunogenic composition so as to achieve the desired level of protection. The individual doses can be administered in ovo and/or post-hatch.

As discussed above, it will be apparent to those skilled in the art that when treating a plurality of birds (such as in commercial poultry production), the effectiveness of the dose and/or the immunogenic composition can be assessed by evaluating the effects of vaccination on the flock as a whole. In other words, an effective immunizing dose or an effective vaccine for the flock as a whole may nonetheless not induce an immune response and/or provide sufficient protection against disease in individual birds.

The terms “vaccination” or “immunization” are well-understood in the art, and are used interchangeably herein. For example, the terms vaccination or immunization can be understood to be a process that increases a subject's immune reaction to antigen (by providing an active immune response), and therefore its ability to resist, overcome and/or recover from infection (i.e., a protective immune response).

The terms “protective immunity” or “protective immune response,” as used herein, are intended to mean that the host animal mounts an active immune response to the immunogenic composition and/or that the immunogenic composition provides passive immunity, such that upon subsequent exposure or a challenge, the animal is able to resist or overcome infection and/or disease. Thus, a protective immune response will decrease the incidence of morbidity and/or mortality from subsequent exposure to the pathogen among treated birds.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

Models of necrotic enteritis (NE) for assessing efficacy of vaccines and vaccination strategies are known in the art. For example, Hofacre et al. (2003, J. Appl. Poult. Res. 12:60-64) described a model in which chickens were fed a corn soy diet with 26% fishmeal from day 0 to day 14 post-hatch. Fishmeal was removed from the diet at day 14. Birds were challenged with coccidia by oral gavage at day 14, then daily from days 17-19 with C. perfringens by oral gavage. Feed conversion ratio, body weight and scoring of gut lesions were used to assess the presence and severity of necrotic enteritis in challenged birds. NE lesions were assessed on day 22 or day 28, using a scale of 0=none, 1=mild, 2=moderate and 3=marked/severe. Other models can be used to assess vaccine efficacy and vaccine regimens as known in the art. In certain embodiments of the present invention, the administration of a composition of this invention (e.g., an effective amount of a composition of this invention) can result in about a 5, 10, 15, 20, 25, 30, 35, 40, 45, 50, 60, 70, 75, 80, 90, or 100% reduction in gut lesions and/or change in body weight in animals of this model or other known or art-accepted model, as compared to non-immunized or control animals.

In additional embodiments of this invention, the compositions and methods of this invention can be used to induce an antibody response in avians that is at least greater than or equal to about 0.5 antitoxin units/mL (e.g., at least about 0.5, 1.0, 1.5, 2.0, 2.5, 3.0, 3.5, 4.0, 4.5, 5.0, 5.5, 6.0, 6.5, 7.0, 7.5. 8.0, 8.5, 9.0, 9.5 or 10 A.U. of anti-toxin antibody per mL of antisera of the avian).

In further embodiments wherein the compositions and methods of this invention are employed, the percentage of eggs of a population of eggs into which a composition of this invention is delivered into the embryo body can be from about 70% to about 100% (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%) of the total number of eggs in the population of eggs to which the composition is administered.

The present invention also encompasses immunogenic compositions that induce an active and/or passive immune response against C. perfringens (including types A and/or C), and which optionally can be used to protect a bird against C. perfringens infection and/or to protect against necrotic enteritis as described in more detail above.

The immunogenic compositions of the invention comprise, consist essentially of, and/or consist of an agent(s) that induces an immune response against Clostridium. The immune agent of Clostridium can be a replicating antigen and/or a nonreplicating antigen. The replicating and nonreplicating antigens of this invention can be delivered in ovo to the amnion, to the embryo and/or to both the amnion and embryo.

Furthermore, the immunogenic compositions of the invention comprise, consist essentially of, or consist of an effective immunizing dose of a Clostridium immunizing agent in a pharmaceutically acceptable carrier. In representative embodiments, the immunogenic composition can be formulated with Clostridium toxoids and/or bacterins. According to this embodiment, the immunogenic composition optionally further comprises an adjuvant (see below). Toxoids are inactivated toxins, and can be derived from Clostridium toxins, including Toxoids are inactivated toxins, and can be derived from Clostridium toxins, including those derived from C. perfringens, including alpha toxin, beta toxin, beta 2 toxin, enterotoxin, epsilon toxin, iota toxin, kappa toxin, lambda toxin, and/or theta toxin; those derived from C. sordellii, including hemorrhagic toxin and/or lethal toxin; those derived from C. difficile, including A toxin (enterotoxin) and B toxin (cytopathic toxin); those derived from C. septicum, including alpha toxin; those derived from C. novyi, including alpha toxin and/or beta toxin; and/or those derived from C. botulinum, including toxin type C. Methods of producing toxoids are known in the art and include, for example, formaldehyde or heat treatment of toxin (see, e.g., Walker, (1992) Vaccine 10:977-990). Bacterins are bacterial cellular components and can be derived from a Clostridium species, such as, for example, from C. perfringens types A and/or C. perfringens type C. C. perfringens toxoid vaccines are known in the art (see, e.g., U.S. Pat. No. 4,292,306 to Zemlyakova).

In other embodiments, the immunogenic composition comprises, consists essentially of, or consists of a killed (i.e., nonreplicating) Clostridium bacterium (i.e., a bacterin), optionally in a water-in-oil-in-water emulsion (see, e.g., U.S. Pat. No. 5,817,320 to Stone describing in ovo immunization of avian embryos with oil emulsion vaccines, the entire contents of which are incorporated by reference herein), and/or a pharmaceutically acceptable carrier. In other embodiments, the immunogenic composition comprises, consists essentially of, or consists of killed Clostridium and an adjuvant (e.g., an aluminum derived adjuvant such as aluminum hydroxide, a saponin such as Quil-A including QuilA QS21, or an oil such as complete or incomplete Freund's), optionally in a water-in-oil-in-water emulsion and/or a pharmaceutically acceptable carrier.

As a further alternative, the immunogenic composition comprises, consists essentially of, or consists of a replicating immune agent of Clostridium, e.g., live C. perfringens, which is generally a live attenuated (i.e., with reduced virulence) C. perfringens. (See, for example PCT Publication No. PCT WO 2005/053737, the entire contents of which are incorporated by reference herein, for teachings on the production of live attenuated bacteria for vaccine use.) Methods of producing attenuated bacteria are known in the art and include without limitation: irradiation, chemical treatment, serial passage in culture, and the like. In certain embodiments of the invention, live Clostridium bacteria (e.g., C. perfringens) are administered in the presence of an agent that protects the subject from the pathological effects of the organism, for example, by co-administration of a neutralizing factor as described in U.S. Pat. No. 6,440,408 to Thoma et al., or interferon as described in U.S. Pat. No. 6,506,385 to Poston et al. Optionally, the Clostridium and the neutralizing factor and/or interferon are administered in the same formulation.

Further examples of immunogenic compositions include immunogenic compositions comprising, consisting essentially of, or consisting of antitoxins (i.e., antibodies that provide passive immunity against Clostridium alpha and/or beta toxins; see, e.g., U.S. Pat. No. 5,719,267 to Carroll et al.), antigenic peptides that induce an immune response against Clostridium (including C. perfringens toxins; see e.g., U.S. Pat. Nos. 5,817,317 and 5,851,827 to Titball et al.; U.S. Pat. No. 6,610,300 to Segers et al.; U.S. Pat. No. 5,695,956 to McClane et al.), and recombinant vaccines that comprise a carrier nucleic acid (e.g., a plasmid or virus) that delivers a nucleic acid encoding an antigenic peptide(s) or protein(s) that induces an immune response against Clostridium.

In representative embodiments, the immunogenic composition comprises, consists essentially of, or consists of a recombinant alpha and/or beta toxin of Clostridium, for example, the alpha toxins having the amino acid sequence as shown in SEQ ID NO:2 [full-length sequence of 370 amino acids; GenBank Accession No. 1GYGB (GI:21730290), the coding sequence of which is provided herein as SEQ ID NO:1], SEQ ID NO:4 (Cpa₂₄₇₋₃₇₀; amino acids 247-370 of SEQ ID NO:2; the coding sequence of which is provided herein as SEQ ID NO:3), SEQ ID NO:6 (amino acids 1-278 of SEQ ID NO:2; the coding sequence of which is provided herein as SEQ ID NO:5), SEQ ID NO:8 (Cpa₂₆₁₋₃₀₀; amino acids 261-300 of SEQ ID NO:2 the coding sequence of which is provided herein as SEQ ID NO:7) as described herein and/or for example, in U.S. Pat. Nos. 5,817,317 and 5,851,827 to Titball et al., or SEQ ID NO:10 [full length sequence of 398 amino acids, the coding sequence of which is provided herein as SEQ ID NO:9]. In some embodiments, the toxin of the present invention is an immunogenic composition comprising amino acids 1-278 (SEQ ID NO:6) of the 370 amino acid sequence (SEQ ID NO:2) of the alpha toxin of C. perfringens.

Further examples of toxins (including immunogenic fragments thereof) that can be used in the compositions and methods of this invention include but are not limited to, C. perfringens toxins [e.g., alpha toxin, Accession number CAA35186 (Saint-Joanis et al. Mol. Gen. Genet. 219(3):453-460 (1989)0; beta toxin, Accession number CAA58246 (Steinthorsdottir et al. FEMS Microbiol. Lett. 130(2-3):273-278 (1995)); beta 2 toxin, Accession number NP_{—}150010 (Shimizu et al. Proc. Natl. Acad. Sci. U.S.A. 99(2):996-1001 (2002)); enterotoxin, Accession number BAE79112 (Miyamoto et al. J. Bacteriol. 188(4):1585-1598 (2006)); epsilon toxin, Accession number AAA23236 (Havard et al. FEMS Microbiol. Lett. 97:77-82 (1992); iota toxin, Accession number CAA51959 (Perelle et al. Infect. Immun. 61(12):5147-5156 (1993)); kappa toxin, Accession number NP_{—}561089, Shimizu et al. Proc. Natl. Acad. Sci. U.S.A. 99(2):996-1001 (2002)); lambda toxin, Accession number CAA35187 (Saint-Joanis et al. Mol. Gen. Genet. 219(3):453-460 (1989)); and theta toxin, Accession number NP_{—}561079 (Shimizu et al. Proc. Natl. Acad. Sci. U.S.A. 99(2):996-1001 (2002))], C. difficile toxins [e.g., A toxin, accession number A37052 (Wren et al. FEMS Microbiol. Lett 70:1-6(1990)); and B toxin, Accession number CAA43299 (von Eichel-Streiber et al. Mol. Gen. Genet. 233(1-2): 260-268 (1992))], C. septicum toxins [alpha toxin, Accession number MB32892 (Ballard et al. Infect. Immun. 63(1):340-344 (1995))] and C. novyi toxins [e.g., alpha toxin, Accession number MB27213 (Ball et al. Infect. Immun. 61(7):2912-2918 (1993))].

The terms “toxin,” “alpha toxin,” “beta toxin,” “epsilon toxin,” (or a like term), etc., as used herein include the full-length toxin as well as antigenic or immunogenic peptides or antigenic or immunogenic variants (e.g., attenuated) thereof that induce an immune response (optionally, a protective immune response) against Clostridium in the subject. In particular embodiments, an antigenic peptide comprises at least about 6, 8, 10, 12, 15, 18, 20, 25, 30, 50, 75 or 100 or more contiguous amino acids of the full-length toxin (see, e.g., the full-length alpha toxin sequence as shown in SEQ ID NO:2 in U.S. Pat. Nos. 5,817,317 and 5,851,827 and in SEQ ID NO:2 herein).

It is also understood that the immunogenic fragments of this invention can be combined in any order or amount. For example, fragment 1-10 can be combined with fragment 10-20 to produce a fragment of amino acids 1-20. As another example, fragment 1-20 can be combined with fragment 50-60 to produce a single fragment of this invention having 31 amino acids (AA 10-20 and AA 50-60). Also fragments can be present in multiple numbers and in any combination in a fragment of this invention. Thus, for example, fragment 1-150 can be combined with a second fragment 1-150 and/or combined with fragment 400-500 to produce a fragment of this invention.

In some embodiments, an antigenic or immunogenic fragment of a Clostridium toxin of this invention can comprise, consist essentially of and/or consist of the amino terminal domain of C. perfringens alpha toxin (amino acids 1-246 of SEQ ID NO:2), the carboxy terminal domain of C. perfringens alpha toxin (amino acids 256-370 of SEQ ID NO:2) and/or the fragment between these domains (amino acids 247-255 of SEQ ID NO:2) in any combination and with any amount of overlap in amino acid sequence that results in a fragment having immunogenic activity. This language is intended to encompass all possible toxin peptides and fragments as if explicitly set forth herein (e.g., any peptide or fragment comprising at least about 6, 8, 10, 12, 15, 18, 20, 25, 30, 50, 75 or 100 or more contiguous amino acids of the full-length alpha toxin sequence as shown in SEQ ID NO:2 in U.S. Pat. Nos. 5,817,317 and 5,851,827 and in SEQ ID NO:2 and SEQ ID NO:10 herein). In particular embodiments, the antigenic peptide lacks an amino acid sequence having phospholipase C and/or sphingomyelin hydrolyzing activity (e.g., an antigenic alpha toxin peptide can lack amino acids 1-240). The location of some C. perfringens alpha toxin epitopes has been determined (see, e.g., Logan et al., (1992) Infection and Immunity 59:4338-4382, the entire contents of which are incorporated by reference for teachings of alpha toxin epitopes).

Additional examples of recombinant Clostridium toxins that can be employed in the methods of this invention include, but are not limited to, a Clostridium perfringens beta toxin or an immunogenic fragment thereof, wherein the beta toxin has the amino acid sequence as set forth in SEQ ID NO:11. The beta toxin of SEQ ID NO:11 can further comprise a mutation at amino acid 62, 182, 197 or in one of the regions between amino acid numbers 80-103, 145-147, 281-291, 295-299 or downstream of amino acid position 292 (as described in U.S. Pat. No. 6,610,300, the entire contents of which are incorporated by reference herein), whereby the resulting toxin or fragment thereof has immunogenic activity.

The nucleic acid and amino acid sequences of C. perfringens alpha and beta toxins are known in the art, see, e.g., GenBank Accession Nos. DQ202275; NP_{—}560952; NC_{—}003366; AY823400; AY277724; AF204209; X17300; X13608; L43548; L43547; L77965 and L13198. See also, Sheedy et al., Highly Conserved Alpha-Toxin Sequences of Avian Isolates of Clostridium perfringens, J. Clin. Microbiol. 42:1345-1347 (2004) presenting an analysis of the alpha toxin sequences of 25 chicken-derived C. perfringens strains.

In further embodiments of this invention, the Clostridium toxin can be an epsilon (ε) toxin of C. perfringens, having an amino acid sequence as set forth in SEQ ID NO:12 (328 amino acids; or SEQ ID NO:13. In further embodiments, the ε toxin can comprise the amino acid sequence of SEQ ID NO:13, wherein residue 2 is a proline, as described in U.S. Pat. No. 6,403,094, the entire contents of which are incorporated by reference herein.

In certain embodiments, the present invention provides a method of immunizing an avian subject against infection by Clostridium, comprising administering to the avian subject an effective immunizing dose of a Clostridium bacterin-toxoid composition by in ovo injection during the final quarter of incubation. The methods of this invention can further comprise the step of administering a booster dose of the Clostridium bacterin-toxoid composition to the avian subject post hatch. The Clostridium species of this invention can include, but is not limited to Clostridium perfringens. In particular embodiments of this invention, the composition can comprise a Vision CD® vaccine. In particular embodiments wherein the subject is a chicken, the bacterin-toxoid composition can be administered into the amniotic fluid via a 20 g, 1.0 inch needle at day 18 of incubation or a 22 g, 1.0 inch needle during day 18 of incubation.

In additional embodiments of this invention, methods are provided of immunizing an avian subject against infection by Clostridium, comprising administering to the avian subject an effective immunizing dose of a recombinant toxin or immunogenic fragment thereof of Clostridium by in ovo injection during the final quarter of incubation. In some embodiments, these methods can further comprise the step of administering a booster dose of the recombinant toxin or immunogenic fragment thereof to the avian subject post hatch. In particular embodiments, the composition employed in these methods can comprise an adjuvant, which can be Quil A and incomplete Freund's adjuvant. In embodiments wherein the subject is a chicken, the bacterin-toxoid composition can be administered into the embryo body via a 23 g, 1.25 inch needle during day 19 of incubation.

In yet further embodiments, when the subject is a chicken, a toxin or immunogenic fragment thereof of this invention can be administered into the embryo body via a 20 g, 1.5 inch needle during day 19 of incubation. Also, the dosage range of a toxin (e.g., an alpha toxin) or immunogenic fragment thereof and/or other subunit protein or glycoprotein or other type of biological molecule used as a vaccine of this invention can be from about 1 μg to about 1000 μg per dose, with an exemplary range of about 55 μg to about 60 μg per dose. For compositions of this invention comprising inactivated virus, the virus concentration per dose can be about 10³ EID₅₀/TCID₅₀ to about 10¹² EID₅₀/TCID₅₀ (EID=egg infectious dose; TCID=tissue culture infectious dose). In embodiments comprising activated virus, the virus concentration per dose can be about 10^{0.1} EID₅₀/TCID₅₀ to about 10¹² EID₅₀/TCID₅₀ In particular embodiments of this invention, the toxin of this invention comprises, consists essentially of and/or consists of the amino acid sequence of SEQ ID NO:2, 4, 6, 8 or 10, including any combination thereof.

As contemplated herein, in some embodiments of the present invention, the composition of this invention further comprises an adjuvant, which in particular embodiments, can be an adjuvant such as an aluminum derived adjuvant (e.g., aluminum hydroxide), a saponin (e.g., Quil-A including QuilA QS21), or an oil (such as Complete or Incomplete Freund's adjuvant), in any combination. Additional examples of adjuvants that can be employed in any of the methods of the inventions described herein are provided herein.

In representative embodiments, the immunogenic composition of this invention comprises, consists of, of consists essentially of a C. perfringens enterotoxin (CPE), beta-2 toxin, epsilon toxin, kappa toxin, lambda toxin, theta toxin, and/or iota toxin, optionally in addition to a C. perfringens alpha and/or beta toxin.

In other representative embodiments, the immunogenic composition comprises, consists essentially of, or consists of a toxoid or toxoid/bacterin. The bacterin can be a C. perfringens type A and/or type C bacterin. For example, an exemplary immunogenic composition comprises, consists essentially of, or consists of an alpha toxoid and a C. perfringens type A bacterin. Optionally, the immunogenic composition further comprises an adjuvant such as an aluminum derived adjuvant (e.g., aluminum hydroxide), a saponin (e.g., Quil-A including QuilA QS21), or an oil (such as Complete or Incomplete Freund's adjuvant).

A representative immunogenic composition of the invention comprises, consists essentially of, or consists of an effective immunizing dose of a C. perfringens immunizing agent in a water-in-oil-in-water emulsion (see, e.g., U.S. Pat. No. 5,817,320 to Stone), optionally in a pharmaceutically acceptable carrier.

The immunogenic composition can optionally comprise two or more agents that induce an immune response against C. perfringens (e.g., any combination of the agents described above).

In particular embodiments, the agent that induces an immune response against C. perfringens (e.g., a toxoid, bacterin, attenuated C. perfringens, and/or toxin and the like) is an avian-derived, optionally a chicken-derived, strain of C. perfringens.

As used herein, the term “consists essentially of” (and grammatical variants) means that the immunogenic composition comprises no other material immunogenic agent other than the indicated agents. The term “consists essentially of” does not exclude the presence of other components such as adjuvants, immunomodulators, and the like.

By “pharmaceutically acceptable” it is meant a material that is not biologically or otherwise undesirable, i.e., the material may be administered to a subject without causing appreciable undesirable biological effects. Thus, such a pharmaceutical composition may be used, for example, to prepare compositions for immunization. Physiologically and pharmaceutically acceptable carriers may contain other compounds including but not limited to stabilizers, salts, buffers, adjuvants and/or preservatives (e.g., antibacterial, antifungal and antiviral agents) as are known in the art. The pharmaceutically acceptable carrier need not be sterile, although it generally will be for in ovo administration to avian embryos.

In particular embodiments, the immunogenic composition further comprises an immune stimulant. Alternatively, the immune stimulant can be administered to the subject in a separate formulation. Immune stimulants that can be used in the present methods include, but are not limited to, cytokines, growth factors, chemokines, supernatants from cell cultures of lymphocytes, monocytes, or cells from lymphoid organs, cell preparations or cell extracts (e.g., fixed Staphylococcus aureus or lipopolysaccharide preparations), mitogens, or adjuvants, including low molecular weight pharmaceuticals. Immune stimulants can be administered in ovo at any time during incubation. Optionally, the immune stimulant and the agent that induces an immune response against C. perfringens are administered concurrently, optionally in the same formulation.

As used herein, the word “concurrently” means sufficiently close in time to produce a combined effect (that is, concurrently can be simultaneously, or it can be two or more events occurring within a short time period before and/or after each other).

Any suitable vaccine adjuvant can be used according to the present invention, including chemical and polypeptide immunostimulants that enhance the immune system's response to antigens. Adjuvants include but are not limited to an aluminum derived adjuvant (e.g., aluminum hydroxide), aluminum phosphate, plant and animal oils (e.g., incomplete or complete Freund's), saponin (e.g., Quil-A including QuilA QS21), Spur® (Intervet), and the like. Representative adjuvants of this invention include but are not limited to an aluminum salt such as aluminum hydroxide gel (alum), aluminum phosphate, or algannmulin, but may also be a salt or mineral gels of calcium, magnesium, iron or zinc, or may be an insoluble suspension of acylated tyrosine, or acylated sugars, cationically or anionically derivatized polysaccharides, or polyphosphazenes, or saponins such as Quil-A, or oil emulsions such as water-in-oil and water-in-oil-in water or complete or incomplete Freund's or any combination thereof.

The immunogenic composition can optionally contain one or more stabilizers. Any suitable stabilizer can be used, including carbohydrates such as sorbitol, mannitol, starch, sucrose, dextrin, or glucose; proteins such as albumin or casein; and buffers such as alkaline metal phosphate and the like.

It is often convenient to immunize a bird against multiple diseases in a single course of treatment. Thus, in particular embodiments, the immunogenic composition comprises one or more additional agents that induce an immune response against other avian pathogens (e.g., viral, bacterial or fungal), optionally immunizing agents that produce a protective immune response. For example, the immunogenic composition can further comprise an immunizing agent against coccidiosis (i.e., Eimeria), infectious bursal disease, Marek's disease, Newcastle disease, avian influenza, fowl pox, avian reovirus, avian metapneumovirus, avian adenovirus, infectious bronchitis, Salmonella spp., Camplyobacter spp., Pasteurella spp., Hemophilus paragallinarum and/or Mycoplasma spp. Avian vaccines suitable for in ovo or post-hatch use are known in the art and are commercially available (e.g., Bursaplex™ vaccine for bursal disease; Newplex™ vaccine for Newcastle disease, and Inovocox™ vaccine for coccidiosis, all available from Embrex, Inc., and Marek's HVT-SB-1 vaccine for Marek's disease, available from Merial). Immunogenic compositions comprising vaccine agents against both coccidiosis (i.e., Eimeria) and necrotic enteritis (i.e., C. perfringens) are particularly advantageous because Eimeria exposure is known to increase the susceptibility of birds to necrotic enteritis by perturbing the gastrointestinal environment.

Thus, as a further aspect, the invention encompasses methods of co-administering an immunogenic composition that comprises, consists essentially of, or consists of an effective immunizing dose of a C. perfringens immunizing agent and an effective immunizing dose of an immunizing agent that provides protection against one or more other avian diseases (as described above). The multiple immunizing agents can be provided in a single formulation or can be administered concurrently or sequentially in any order in separate formulations. As discussed above, this aspect of the invention is particularly suited to co-administration of coccidiosis and necrotic enteritis vaccines.

In another representative embodiment, the avian subject is first immunized against necrotic enteritis and is then immunized against coccidiosis or vice versa. The immunizations can both occur in ovo, both can occur post-hatch, or one can be in ovo and one post-hatch. For example, in one illustrative embodiment, the avian subject is immunized against coccidiosis in ovo and then is immunized against necrotic enteritis after hatch.

The invention can also be practiced to administer a C. perfringens immunizing agent in ovo or post-hatch in conjunction with “in ovo feeding” (see, U.S. Pat. No. 6,592,878; incorporated by reference herein in its entirety) of the avian subject. For example, according to certain embodiments, a C. perfringens immunizing agent and a nutrient formulation and/or enteric modulator are administered to an avian subject in ovo, optionally by delivery to the amnion. Optionally, vaccines against other infectious agents are administered in ovo and/or post-hatch as well (as described above). The C. perfringens immunizing agent and the nutrient formulation and/or enteric modulator can be administered concurrently, in the same or separate compositions, and/or can be administered sequentially in any order.

Further embodiments of the present invention can include a composition comprising an antigen selected from the group consisting of a C. perfringens alpha toxoid, an antigenic fragment of a C. perfringens alpha toxoid, an inactive antigenic fragment of a C. perfringens alpha toxin, and any combination thereof; wherein one or more doses of about 0.1 to about 1.0 mL (e.g., 0.1, 0.2, 0.3, 0.4, 0.5, 0.6, 0.7, 0.8, 0.9, or 1.0) per dose of the composition is sufficient to induce at least 0.5 antitoxin units (A.U.) of anti-alpha toxin antibody per mL of antisera of an avian (e.g., chicken) vaccinated with the vaccine. In some embodiments, the composition can induce at least about 0.5, 1.0, 1.5, 2.0, 2.5, 3.0, 3.5, 4.0, 4.5, 5.0, 5.5, 6.0, 6.5, 7.0, 7.5. 8.0, 8.5, 9.0, 9.5 or 10 A.U. of anti-toxin antibody per mL of antisera of the avian.

As used herein an “antitoxin unit” or “A.U.” of antitoxin antibody per mL of antiserum (which can be used interchangeably with “anti-Toxin Neutralizing Test” units or “TNT” units), is defined by the ability of sera to neutralize the toxic effects of a toxin in a mouse bioassay. In this test, a known amount of toxin, established by international standards as are known in the art, is mixed with serial dilutions of serum from vaccinated animals. The mixture is incubated one hour at room temperature and then injected intravenously into mice. The mice will survive if the toxin is completely neutralized by the sera, otherwise they die. The antitoxin units or titer is determined as the reciprocal of the highest dilution of sera that neutralized the toxin.

In further embodiments, the composition can comprise an antigen in a cell-free preparation. In other embodiments, the antigen can be an alpha toxoid in a C. perfringens alpha toxoid supernatant. In certain embodiments, the composition can comprise, consist essentially of and/or consist of an antigen that can be a C. perfringens Type A alpha toxoid and/or a C. perfringens Type C alpha toxoid. In some embodiments, the composition cf this invention can comprise C. perfringens beta toxin, C. perfringens beta 2 toxin, C. perfringens enterotoxin, C. perfringens epsilon toxin, C. perfringens iota toxin, C. perfringens kappa toxin, C. perfringens lambda toxin, C. perfringens theta toxin, C. sordellii hemorrhagic toxin, C. sordellii lethal toxin, C. difficile A toxin, C. difficile B toxin, C. septicum alpha toxin, C. novyi alpha toxin, C. novyi beta toxin and/or any combination thereof. Such a composition can further comprise, consist essentially of and/or consist of one or more viral antigens, one or more bacterial antigens, and/or one or more parasitic antigens as described herein.

The present invention is explained further in the following non-limiting examples. In these Examples, “.μL” means microliters, “.μg” means micrograms, “mL” means milliliters, “cc” means cubic centimeters, “mm” means millimeters, “mM” means concentration in millimoles, “mg” means milligrams, “° C.” means degrees Celsius and E18 and E19 mean embryonation days 18 and 19, respectively.

### EXAMPLES

### Example I

### Immune Response Following in Ovo Vaccination with Commercially Available Clostridium perfringens Type C&D Vaccines (Siteguard G & Vision CD)

Experimental Design

Broiler eggs were manually in ovo injected with commercially available Clostridium perfringens toxoid (Siteguard® G) and bacterin-toxoid vaccines (Vision® CD® vaccine). Hatched birds were grown out to measure antibody responses. Site of Injection evaluation was performed. At day 0 (hatch) select treatment groups received a post hatch vaccination. All birds were housed in cages (5 birds/cage). Each cage was supplied with a diet of Normal Broiler Starter. On day 14 birds were switched over to Broiler Grower Feed. Birds were bled and serum was tested for an antibody response by serum-toxin neutralization assay.

Materials & Methods

Injection Material (Siteguard® G):

Siteguard® G (adjuvant unknown) is a C. perfringens type C & D toxoid vaccine produced by Schering-Plough. It protects sheep and cattle from diseases caused by type C & D toxins. For vaccination, 4.0 mL (cattle) or 2.0 mL (sheep) of the vaccine is administered subcutaneously (SQ) or intramuscularly (IM). Booster vaccinations are administered three to four weeks post initial vaccination and annually.

Injection Material (Vision® CD®):

Vision® CD® (with the proprietary adjuvant ‘Spur®’) is a C. perfringens type C & D bacterin-toxoid vaccine produced by Intervet. It protects cattle, sheep, and goats from enterotoxemia caused by C. perfringens type C & D. For vaccination, 2.0 mL of the vaccine is administered subcutaneously into the animal (cattle, sheep, or goat). Three to four weeks post initial vaccination, the animal receives an additional 2.0 mL (SQ) and is re-vaccinated annually thereafter.

Injection Protocol:

On E19, broiler eggs were injected with test materials targeting either the amniotic fluid or embryo body of each egg. In addition, at day 0 post hatch, some in ovo and non-in ovo injected treatment groups received a vaccination or booster immunization of test materials. For the post-hatch vaccination or booster immunization, 0.5 mL of vaccine was administered by subcutaneous injection in the back of the neck.

Site of Injection:

At the time of injection, eggs allotted for site of injection evaluation were injected with dye. Eggs were then euthanized and necropsied for site of injection evaluation. Site of injection was analyzed by likelihood ratio chi-square. (Table 1)

Bleeding:

At days 7, 14, 21, and 28 post hatch, blood was collected from each individual bird and pooled in individual vacutainers (per treatment group). At days 7, 14 and 21; ≦0.5 mL of blood was collected via either the wing or jugular. At day 28, ≧0.5 mL of blood was collected via cardiac puncture. Blood was then incubated at room temperature for one hour. Then the blood samples were placed in a table top centrifuge at 2400 RPMs for 10 minutes. Once centrifugation was complete, serum was removed from each blood sample and stored in a 96 well storage plate (2-8° C. or −70° C.) for future immune response evaluations.

Clostridium perfringens Type C (Beta) Toxin Neutralization Testing in Mice: Sample Preparation for Mouse Inoculation

A. Materials

1. Clostridium perfringens Type C (beta) toxin—CVBL Lot no. IRP513(04)
2. Clostridium perfringens Type C (beta) antitoxin—CVBL Lot no. IRP486 Receipt
3. Diluent—1% peptone, 0.25% NaCl pH 7.2, BBL Lot no. 051006, NB ref.—NB 140 p. 87
4. Chicken Serum Samples—EMHE1381, NB 140 p. 80
   a. Sample nos. 7, 8, 9, 10A, 10B, 11A, 11B, 12A, 12B, 13A, 14A, 15A
5. Sterile 3 ml and 1.8 ml vials.
   B. Methods:
1. Dilute standard beta antitoxin 1:50 in diluent, 10 mls total.
   a. Thaw beta antitoxin at room temp.
   b. Mix 200 μl of beta antitoxin and 9.8 ml of diluent=1:50.
   c. Hold on ice.
2. Dilute beta toxin 1:120 in diluent, 12 mls total.
   a. Thaw beta toxin at room temp.
   b. Mix 200 μl of beta toxin and 1.8 ml of diluent=1:10
   c. Mix 1 ml of the 1:10 diluted beta toxin with 11 mls of diluent=1:120
   d. Hold on ice
3. Prepare the Lₒ control sample.
   a. Mix 0.5 ml of beta toxin (1:120 dilution) with 0.5 ml of diluent.
   b. Add 1 ml of beta antitoxin (1:50 dilution).
   c. Mix and incubate at room temperature for 1 hour.
   d. Hold samples on ice.
4. Prepare the L₊ control sample.
   a. Mix 0.8 ml of beta toxin (1:120 dilution) with 0.2 ml of diluent.
   b. Add 1 ml of beta antitoxin (1:50 dilution).
   c. Mix and incubate at room temperature for 1 hour.
   d. Hold samples on ice.
5. Prepare the 12 test serum samples.
   a. Mix 3.25 ml of beta toxin (1:120 dilution) with 3.25 ml of diluent.
   b. To each of the twelve 1.8 ml tubes, add 0.5 ml of toxin from step 5.a.
   c. Label each tube with sample number and treatment group number.
   d. Add 0.5 ml of each undiluted chicken serum to the appropriately labeled tube.
   e. Mix and incubate at room temperature for 1 hour.
   f. Store samples on ice.
6. All unused samples are stored at 2-7° C.
   Pre-Study Activities

Seventy-eight female Swiss white (CD-1) mice (16-20 grams body weight) were purchased for use in the study. Mice were shipped from the vendor (Charles River Laboratories) and transported to the Clinical Testing Facility.

Mice were housed in cages placing 2 mice per cage for the chicken serum groups and 4 cages of 5 mice per cage for the control groups. The mice were held for an acclimation period of 5 days prior to initiation of the study on Day 0. Mice were housed and cared for according to standard operating procedures and fed a standard laboratory diet and offered water ad libitum.

Day 0

Mice were examined for normal health and appearance and placed on test enrolling seventy-six mice. Two mice were not enrolled on study and were euthanized. Each mouse was injected intravenously (IV) with a 26 g×⅜ needle in the tail vein according to treatment groups described under STUDY DESIGN. Mice were monitored twice daily for signs of shock, pain or distress as evidenced by the following:

Lethargy

Huddling

Rough/ruffled hair coat

Hunched posture

Ataxia

Anorexia or inability to reach food and water

Moribund mice were euthanized via CO₂ overdose according to standard operating procedures.

Day 1 (Approximately 24 Hours Post Inoculation)

Mice were observed and number of mortalities recorded.

Results

A positive specific antibody response was detected in sera from birds vaccinated in ovo (embryo body-targeted) with a commercial C. perfringens bacterin-toxoid vaccine with and without a post-hatch boost using the serum-toxin neutralization assay. These data indicate that administration of a C. perfringens bacterin-toxoid vaccine in ovo elicits partial protection and in ovo administration followed by a post-hatch boost confers full protection against C. perfringens. (Table 2)

### Example II

### Immune Response Following in Ovo Vaccination with an Experimental Vaccine Preparation Containing Recombinant Alpha Toxin

Experimental Design

The above study was conducted to determine if a humoral (antibody) immune response could be detected in broiler birds following in ovo, in ovo+post hatch or post hatch vaccinations with a Clostridium perfringens recombinant alpha toxin (SEQ ID NO:6) (provided by Dr. Glenn Songer, Dept. of Veterinary Science and Microbiology, The University of Arizona), adjuvanted with Incomplete Freund's Adjuvant and Quil-A. The immunization strategy included in ovo embryo body targeting at E18 as well as a day 7 post hatch vaccination. Antibody response was evaluated at 28 days of age.

On E18, Broiler eggs were manually in ovo injected with either control materials (Quil A; Accurate Chemical & Scientific Corporation, Product #AP04991, adjuvant grade, Batch #L77-238) emulsified with Incomplete Freund's Adjuvant (IFA; Rockland, Lot #16235) or C. perfringens recombinant alpha toxin (55 or 60 μg/dose adjuvanted with Quil A+IFA (13 or 15 μg/dose). Site of injection evaluation was performed by injection of dye. At day 0 (hatch) birds were housed in cage units (5 birds/cage). Birds received Normal Broiler Starter. Additionally, some birds were vaccinated on day 7 or 17 according to treatment (0.2 mL of vaccine by subcutaneous injection in the back of the neck). Bird sera were then evaluated for specific antibody response via western blot.

Materials & Methods

Injection:

On E18, broiler eggs were injected with test materials (Clostridium perfringens alpha toxin+vaccine adjuvant) for targeting the embryo body of each egg. In addition, at day 7 or day 17 post hatch, some in ovo and non-in ovo injected treatment groups received a vaccination (Table 3)

Site of Injection (SOI):

At the time of injection, eggs allotted for SOI evaluation were injected with dye. Eggs were then euthanized and necropsied for SOI evaluation. (Table 4)

Sera Collection:

At days 7, 14, 21, and 28 post hatch, blood was collected from each individual bird and placed in individual vacutainers. At days 7, 14, and 21≦0.5 mL of blood was collected via either the wing or jugular vein. At day 28, ≧0.5 mL of blood was collected via cardiac puncture. Blood was then incubated at room temperature for 1 hour. Blood samples were centrifuged and serum was removed and stored in a 96 well storage plate (2-8° C. or −70° C.) for immune response evaluation.

Western Blot Testing:

SDS slab gel electrophoresis was carried out according to the method of Laemmli (Nature 227:680-685 (1970)) as described by O'Farrell (J. Biol. Chem. 250:4007-4021 (1975), second dimension), using a 10% acrylamide slab gel (125 mm length×150 mm width×0.75 mm thickness) overlaid with a 25 mm stacking gel. Electrophoresis was carried out at 12 mAmp for about 3.5 hours or until the bromophenol blue front had migrated to the end of the slab gels. After slab gel electrophoresis, the gel for blotting was placed in transfer buffer (12.5 mM Tris, pH 8.8, 96 mM glycine, 20% MeOH) and transblotted onto a PVDF membrane overnight at 200 mA and approximately 100 volts/two gels. The PVDF membrane was then Coomassie blue stained and dried between sheets of filter paper.

The PVDF membrane was stained with Coomassie Brilliant Blue R-250 and desktop scanned before and after cutting into individual lanes. Each blot lane was placed in a separate container and blocked for two hours in 5% nonfat dry milk in Tween-20 Tris buffered saline (TTBS) and rinsed in TTBS. The blots were then incubated in primary antibody (diluted 1:100 in 2% nonfat dry milk in TTBS) overnight and rinsed 3×10 minutes in TTBS.

The blot lane 1 (positive control) was then placed in secondary antibody [rabbit anti-goat IgG-HRP (Sigma Cat. #A-5420 and Batch #034K4858), 1:5,000 diluted in 2% NFDM in TTBS] for two hours, rinsed 3×10 minutes in TTBS, treated with ECL and exposed to x-ray film.

The remaining blot lanes were then placed individually in secondary antibody [rabbit anti-chicken IgG-HRP (Bethyl Cat. #A30-107P and Batch #A30-107P-3), 1:2,000 diluted in 2% nonfat dry milk in TTBS] for two hours, rinsed 3×10 minutes in TTBS, treated with ECL and exposed to x-ray film.

Results of the western blotting studies are described in Table 5.

Results

A specific antibody response was detected in birds vaccinated in ovo (embryo body-targeted) with recombinant alpha toxin adjuvanted with Quil-A & IFA with and without a post-hatch boost. These data demonstrate that in ovo vaccination with a recombinant alpha toxin can elicit an immune response against C. perfringens alpha toxin in broilers.

### Example III

A commercially available inactivated oil emulsion vaccine for Newcastle disease was purchased from Maine Biological Laboratories. The vaccine was administered in ovo on E18 via the amniotic fluid route or in ovo on E19 via the embryo body route. Site directed administration to the amniotic fluid and embryo body was confirmed by conducting a site of injection analysis using dye on E18 or E19. Site directed administration to the amniotic fluid was accomplished using a blunt needle (Group 2). Site directed administration to the embryo body was done using a sharp 1.25-inch needle (Group 3). Blood serum was collected at 14, 21 and 28 days of age and assayed for antibodies specific to Newcastle disease virus (NDV) using ELISA (Idexx, Inc.). Different individual birds were bled on each blood collection day.

The E18 site of injection analysis indicated that 22/24 eggs were injected in the amniotic fluid, 22/24 in the allantoic fluid, and 0/24 in the embryo body. The E19 site of injection analysis indicated that 7/10 eggs were injected in the embryo body and 3/10 eggs were injected in the amniotic fluid. Table 6 shows the antibody response to Newcastle disease virus following in ovo vaccination of chickens. Table 7 shows percent hatch data.

The study demonstrates that the immune response of the developing embryo is strongly influenced by the in ovo administration site of inactivated oil emulsion Newcastle disease vaccine (Table 6). Embryos vaccinated in the amniotic fluid that surrounds the embryo body did not respond with Newcastle disease specific antibodies. On the other hand, embryos vaccinated directly into the body of the embryo responded with a strong antibody response that increased with age to 28 days. A total of 34 birds were bled over the course of this study and 26/34 were positive for antibodies to Newcastle disease virus (Table 6). 26/34 is 76.5%, which is very similar to the site of injection study where 70% of the embryos immunized on E19 were injected into the embryo body. Percent hatch was within normal ranges for treated and non-treated groups (Table 7).

### Example IV

A commercially available inactivated oil emulsion vaccine for Newcastle disease was purchased from Maine Biological Laboratories. The vaccine was administered in ovo on E19 via the embryo body route or subcutaneous at hatch. Site directed administration to the embryo body was done using a sharp 1.25-inch needle (Group 3). Day of hatch vaccination was done by injecting vaccine subcutaneous in the nape of newly hatched chicks (Group 2). Blood serum was collected at 21 days of age and assayed for antibodies specific to NDV using ELISA (Idexx, Inc.). The results are shown in Table 8.

The data presented in Table 8 show that embryos vaccinated in the body with the Newcastle disease oil emulsion vaccine responded as well as chicks vaccinated by the standard day of hatch route. Percent hatch was within normal ranges for treated and non-treated groups (Table 9).

The data presented in examples III and IV above show that hitting the embryo body is necessary to stimulate an active immune response to an inactivated antigen (in this case Newcastle disease virus). These data also indicate that embryos are not negatively affected by injection into the embryo body with an inactivated antigen in an oil-emulsion adjuvant.

In ovo (prior to hatch) embryo body injection may be accomplished manually using syringe and needle or by an automated injection device also using needles. In the examples given herein, syringe and needle were used manually to apply vaccine to the embryo body or the amniotic fluid (example III only) surrounding the embryo body. To accomplish the embryo body injection, the needle was inserted through a hole in the shell at the air cell end of the egg. The inserted needle passed through the air cell membrane, the allantoic membranes and fluid and finally into the amnion cavity where the embryo body resides. Next the needle penetrated the embryo body and vaccine was deposited. Embryo body injections can occur in numerous sites within the embryo's body and include subcutaneous, intra-dermal, intravenous, intramuscular and intra-abdominal deposition of vaccine, as well as any combination of these sites. Furthermore, embryo body injections can occur in the head, neck, shoulder, wing, back, breast or leg, including any combinations. Embryo body injection does not include exclusive vaccine deposition in the air cell, the allantoic cavity, the amniotic fluid or the albumin.

Embryo body injection in ovo may be done using needle of a length ranging from ¾ inch to up 4 inches and gauges ranging from 15 to 28. Needle tips may range from very sharp (hypodermic) to blunt.

In examples III and IV, Newcastle disease virus vaccine was used as the model antigen. However, any properly formulated oil emulsion vaccine with enough antigenic mass would be expected to be similar to the Newcastle disease vaccine tested. Therefore, inactivated vaccines to infectious bursal disease, avian influenza, infectious bronchitis, chick infectious anemia virus, laryngotracheitis, avian reovirus, adenovirus, rotavirus, astrovirus, inclusion body hepatitis, egg drop syndrome, Escherichia coli, Mycoplasma spp., Salmonella spp., Campylobacter spp, Clostridium spp., Haemophilus spp, Pasteurella spp. can be delivered in ovo directly to the embryo's body according to the methods described herein. Vaccines made from these agents may be whole cell or subunit. Vaccines made from these agents may be produced conventionally in growth media, eggs or tissue culture and/or may be produced by recombinant means according to methods well known in the art. In addition, any disease agent that can be produced to have enough antigenic mass to effectively vaccinate day of hatch chicks when inactivated would also be expected to effectively vaccinate embryos in ovo if delivered directly to the embryo body.

The adjuvant used in the vaccine tested in these examples was a typical commercial oil emulsion. Non-oil emulsion inactivated vaccines with adjuvants other than oil would be expected to produce an active immune response if delivered directly to the embryo body prior to hatch. Adjuvants suitable would include, but are not limited to, mineral gels, polyanions, pluronic polyols, saponin derivatives, lysolecithin and other similar surface active substances, glycosides and all types of oils and combinations thereof.

### Example V

Specific pathogen free (SPF) leghorns were vaccinated in ovo as follows Group 1: phosphate buffered saline (PBS); Groups 2 and 3: 0.3×10⁹ inactivated NDV EID₅₀/dose in PBS; Group 4: 0.3×10⁹ inactivated NDV EID₅₀/dose mixed with an alum depot adjuvant (Imject, Pierce; aluminum hydroxide and magnesium hydroxide); Group 5: a commercial oil emulsion vaccine for NDV. On day 11 of age, Group 3 subjects received a second dose of NDV in PBS by subcutaneous injection. The vaccines given in ovo were targeted to the embryo body and a site of injection analysis using dye was conducted on a separate set of like eggs to estimate the percent of embryos injected directly into the embryo body. The in ovo vaccination was done on day 19 of incubation with a 23 gauge 1.25 inch needle. Fourteen birds per group were placed in cages and grown to 21 days of age.

Serum samples were collected on day 21 of age and tested for IgG antibody to NDV by ELISA (Idexx, Inc.). Serum samples from Groups 2, 4 and 5 were also tested for NDV specific antibody by hemagglutination inhibition (HI) using four HA units. The number of samples tested by Hi differs from those tested by ELISA because with several samples there was not enough serum collected to conduct the HI test. Birds were considered to have shown a measurable antibody response (i.e., seroconverted) to the vaccination if the serum sample had an ELISA titer≧200 or an HI titer of ≧3.0 log₂ (i.e. titer of 1:8).

Results.

The site of injection analysis indicates that embryo body injections accounted for 78% of all injections (Table 10).

The percent hatch and rate of seroconversion to NDV as measured by ELISA are shown in Table 11. In Table 12, the number of birds that seroconverted using the HI test is reported.

From these studies, the following key points were noted. 1) NDV antigen in PBS did not stimulate a measurable antibody response by NDV ELISA, even when the inactivated NDV antigen was given twice as in Group 3 (once in ovo and again on day 11 of age); 2) The NDV-Alum (Group 4) stimulated seroconversion in 8/14 birds when measured by ELISA, while the commercial oil emulsion vaccine (Group 5) stimulated seroconversion in 10/13 birds when measured by ELISA; 3) The NDV-Alum (Group 4) stimulated seroconversion in 12/14 when measured by HI, while the oil emulsion vaccine stimulated seroconversion in 11/11 birds when measured by HI; and 5) The commercial oil emulsion vaccine for Newcastle disease (Group 5) stimulated a stronger antibody response than did the NDV-Alum vaccine (Group 4). Example III shows that in ovo administration of an antigen presented in an oil emulsion depot adjuvant required the vaccine to be delivered to the embryo body to stimulate a measurable antibody response by ELISA. In the present example, in ovo site of injection analysis indicated that 78% of eggs received vaccine directly in the embryo body.

### Example VI

A study was conducted using SPF leghorns to determine if alum depot adjuvant stimulated an immune response when administered in ovo. The groups tested were as follows: Group 1: phosphate buffered saline (PBS) in ovo; Groups 2 and 3: 1.2×10⁹ EID₅₀ β-propiolactone inactivated NDV/dose in PBS in ovo; Group 4: in ovo administration of 1.2×10⁹ EID₅₀ β-propiolactone inactivated NDV/dose mixed with alum at a 30%:70% alum to NDV antigen ratio. The alum used was a commercial solution of aluminum hydroxide and magnesium hydroxide (Imject, Peirce). Group 3 received an additional dose of NDV antigen in PBS on day 11 of age by subcutaneous injection. The in ovo vaccine administration was done on day 19 of incubation using a 23 gauge 1.25 inch needle. The vaccines were targeted in ovo to the embryo body and a site of injection analysis was conducted on a separate set of like eggs to estimate the percent of embryos injected directly into the embryo body. Serum samples were taken on day 21 of age and tested for antibody to NDV by ELISA (Idexx, Inc.). Birds were considered to have shown a measurable antibody response (i.e., seroconverted) if the ELISA had a titer≧200.

Results.

The site of injection analysis indicates that 81% of embryos were injected directly into the embryo body (Table 13). The percent hatch and rate and magnitude of seroconversion against Newcastle diseases virus are shown in Table 14.

These studies provided the following key points: 1) Embryos were injected on E19 and the site of injection data indicated that 81% of embryos were injected in the body of the embryo; and 2) In this study, the alum was mixed with β-propiolactone inactivated NDV in a 30% to 70% ratio and this differed from the study described herein in Example V, in which heat inactivated NDV was mixed at a 50% alum to 50% NDV antigen ratio. The difference in immune responses in the two studies may be due to the differences in NDV antigen used and/or the difference in alum to antigen ratios tested as well as differences in the ELISA used to measure the antibodies.

In example III it was shown that antigen presented in the depot adjuvant oil emulsion requires the vaccine to be delivered to the embryo body in ovo to stimulate a measurable antibody response by ELISA. In this study the site of injection analysis indicated that 81% of embryos were injected in the embryo body, and from these data it would be expected that approximately 11 of the 14 eggs vaccinated in ovo would respond with an antibody response. The actual number that responded was nine of 14.

### Example VII

A commercial oil emulsion Newcastle disease vaccine was given via the in ovo route to broilers. This study determined the ability of broilers to respond to inactivated Newcastle disease virus antigens when delivered in ovo by the amniotic fluid route and the intra-embryo route. Birds were bled at 13, 21, 26 and 35 days of age and antibody titer to NDV was determined using ELISA (Idexx, Inc.). Site of injection analysis was conducted on E18 and E19 using dye.

Hatch data are shown in Table 16. Percent hatch was normal when the oil emulsion vaccine was delivered into the embryo body.

Site of injection (Table 15) was very accurate with greater than 90% of embryos injected by the route indicated for the treatment (Table 16).

The Newcastle disease virus specific antibody response data are shown in Table 17. It can be seen that birds responded to the Newcastle antigen when the vaccine was delivered in ovo into the embryo body or subcutaneous at hatch. When the NDV vaccine was delivered in ovo to the amniotic fluid, there was no antibody response, indicating that the vaccine has to be given to the embryo body to stimulate an appropriate immune response.

The foregoing is illustrative of the present invention, and is not to be construed as limiting thereof. The invention is defined by the following claims, with equivalents of the claims to be included therein.

TABLE 1
Percent Hatch & Site of Injection
Bird Type: Broilers
# Eggs injected/trt group: 40 (100 for SOI)
Embryo Age: E19
Needles: 22 g 1.00″ & 23 g 1.25″
Injection Volume: 1 mL

| | % Hatch | | | | | |
|---|---|---|---|---|---|---|
| | | | Vision CD | | | |
| | | Siteguard G | bacterintoxoid | | | |
| | Control | toxoid | cattle/ | % Hatch | Site of Injection | |
| Injection | Marek's | cattle/ | sheep/ | by Inj. | Amniotic | Embryo |
| Type | Diluent | sheep | goats | Type | fluid | body |
| Negative | N/A | N/A | N/A | 100% | | |
| Control | | | | | | |
| (punched, no | | | | | | |
| inj.) | | | | | | |
| BR-E19 | 95.0% | 75.0% | 82.5% | 84.2% | 1.1% | 97.9%^{b} |
| 23 g 1.25″ | | | | | | |

Eggs that received Vision CD or Siteguard G demonstrated slightly lower overall hatch rates than eggs that received control material (82.5% and 75.0% vs. 95.0%, respectively).

TABLE 2
Serum-toxin neutralization assay results:

| | | | | | # healthy | |
|---|---|---|---|---|---|---|
| Study # | Bird type | Dose | Trt | Description | mice | Result |
| 1 | SPF | — | — | D0 SPF sera | 0/2 | Valid negative |
| | | | | NEG CTL | | control. |
| | | | | | | Neither mouse |
| | | | | | | survived, as expected, |
| | | | | | | indicating specific Abs |
| | | | | | | were not present. |
| 2 | SPF | 1 mL | 4 | D49 sera | 2/2 | Valid positive control. |
| | | | | Hyperimmunized | | Both mice survived as |
| | | | | POS CTL | | expected, indicating |
| | | | | (vaccination + | | specific antibodies |
| | | | | 2 boosts) | | were present. |
| 3 | BR | 1 mL | 7 | D28 sera | 0/2 | No protection |
| | | | | toxoid | | |
| | | | | (Siteguard G) - | | |
| | | | | EMBRYO BODY | | |
| | | | | IN OVO | | |
| | | 1 mL | 8 | D28 toxoid | 0/2 | No protection |
| | | | | (Siteguard G) - | | |
| | | | | EMBRYO BODY | | |
| | | | | IN OVO + | | |
| | | | | post-hatch boost | | |
| | | 1 mL | 11 | D28 sera | 1/2 | Partial protection |
| | | | | bacterin-toxoid | 1/2 | |
| | | | | (Vision CD) - | | |
| | | | | EMBRYO BODY | | |
| | | | | IN OVO | | |
| | | 1 mL | 12 | D28 sera | 2/2 | Complete protection - |
| | | | | bacterin-toxoid | | Satisfactory |
| | | | | (Vision CD) - | | |
| | | | | EMBRYO BODY | | |
| | | | | IN OVO + | | |
| | | | | post-hatch boost | | |

All sera were pooled (˜5 birds/group)
A specific positive response was detected in birds vaccinated in ovo (embryo body-targeted) with the C. perfringens bacterin-toxoid vaccine (Vision CD) followed by a post-hatch boost. Results suggested a low antibody response (partial protection) in birds vaccinated in ovo alone (no post-hatch boost).

No protection was observed in sera from birds vaccinated with a C. perfringens toxoid vaccine (Siteguard).

TABLE 3
Study Summary

| | |
|---|---|
| Bird | Broiler |
| Day of Injection | E18, D7, and D17 |
| Type of | Embryo +/− Post Hatch boost (D7), Post hatch only |
| Injections | (D7) w/boost (D17) |
| Control | Non-injected (punched) |
| Test Materials | Inactivated Recombinant Alpha Toxin (SEQ ID NO: |
| | 6), adjuvanted with Quil A + Incomplete Freund's |
| | Adjuvant |
| Toxin Dose | 55.2-60 μg/0.2 mL |

TABLE 4
Percent Hatch & Site of Injection
Bird Type: Broiler
#Eggs injected/trt group: 15-25 (100 for SOI)
Embryo Age: E18
Needles: 20 G 1.5″
Injection Volume: 0.2 mL

| | % Hatch | | | | |
|---|---|---|---|---|---|
| | | | Recomb | | |
| | | | Alpha | | |
| | | | Toxin + | Site of Injection | |
| Inj. | Non- | Quil-A + | Quil-A + | Amniotic | Embryo |
| Type | Inj. | ICFA | ICFA | fluid | body |
| Non-inj. | 100% | | | | |
| Neg. control | | | | | |
| 20 g 1.5″ | | 67% | 96% | 27.84% | 72.16% |
| 0.2 mL | | | | | |

Percent hatch of 96% was achieved following in ovo vaccination with the recombinant alpha toxin. 72.16% embryo targeting was achieved using the 20 g 1.5″ needle.

TABLE 5
Detection of specific antibody response via western blot.

| Lane | Sample ID | Description | Result |
|---|---|---|---|
| 1 | Positive control - | Positive control | + |
| | goat anti alpha | pooled sera | |
| 2 | Negative control - | Negative Control RP-967 | − |
| | D0 SPF | pooled sera | |

D28 sera

| | | | |
|---|---|---|---|
| 3 | #1 | Trt 4A - in ovo negative vehicle | − |
| | | control Pooled sera from 3 birds | |
| 4 | #2 | Trt 5A - in ovo embryo-vaccinated | + |
| | | Pooled sera from 3 birds | |
| 5 | #3 | Trt 5A - in ovo embryo-vaccinated | + |
| | | Pooled sera from 3 birds | |
| 6 | #4 | Trt 6A - in ovo embryo + post-hatch | + |
| | | vacc | |
| | | Pooled sera from 3 birds | |
| 7 | #5 | Trt 6A - in ovo embryo + post-hatch | + |
| | | vacc | |
| | | Pooled sera from 3 birds | |
| 8 | #6 | Trt 2A - post-hatch positive control | + |
| | | Pooled sera from 3 birds | |

Positive and negative controls performed as expected, demonstrating test validity.

A specific antibody response was detected in birds vaccinated in ovo (embryo body-targeted) with the recombinant alpha toxin formulation with and without a post-hatch boost.

TABLE 6
Antibody response of chickens to Newcastle diseases virus
following site directed in ovo administration of an inactivated oil
emulsion Newcastle disease vaccine

| | | Day 14 | | Day 21 | | Day 28 | |
|---|---|---|---|---|---|---|---|
| | | of age | | of age | | of age | |
| | Vaccine | Mean | # pos./ | Mean | # pos./ | Mean | # pos./ |
| Gp | Route | titer | # tested¹ | titer | # tested | titer | # tested |
| 1 | Non- | 7 | 0/10 | 1 | 0/12 | 23 | 0/12 |
| | vaccinated | | | | | | |
| 2 | NDV vaccine | 1 | 0/10 | 1 | 0/12 | 1 | 0/12 |
| | in ovo | | | | | | |
| | amniotic fluid | | | | | | |
| 3 | NDV vaccine | 909 | 7/10 | 3262 | 8/12 | 7819 | 11/12 |
| | in ovo | | | | | | |
| | Embryo body | | | | | | |

¹number of birds positive for antibodies to Newcastle disease/number of birds tested.

TABLE 7
Percent hatch following site directed in ovo administration of an
inactivated oil emulsion Newcastle disease vaccine

Percent hatch data

| | Vaccine | # hatched/ | |
|---|---|---|---|
| Group | Route | # transferred | Percent hatched |
| 1 | Non-vaccinated | 48/50 | 96% |
| 2 | NDV vaccine | 51/60 | 85% |
| | in ovo | | |
| | amniotic fluid | | |
| 3 | NDV vaccine | 48/60 | 80% |
| | in ovo | | |
| | Embryo body | | |

TABLE 8
Antibody response of chickens to Newcastle disease virus
following intra-embryo in ovo administration or subcutaneous
administration at day of hatch of an inactivated oil emulsion
Newcastle disease vaccine

| | |
|---|---|
| Vaccine | Day 21 of age |

| Group | Route | Mean titer | # pos./# tested¹ |
|---|---|---|---|
| 1 | Buffer | 1 | 0/12 |
| | in ovo | | |
| | Embryo body | | |
| 2 | NDV vaccine | 3785 | 10/12 |
| | Subcutaneous | | |
| | at hatch | | |
| 3 | NDV vaccine | 3261 | 12/12 |
| | in ovo | | |
| | Embryo body | | |

¹number of birds positive for antibodies to Newcastle disease/number of birds tested

TABLE 9
Percent hatch following intra-embryo in ovo administration of
an inactivated oil emulsion Newcastle disease vaccine

Percent hatch data

| | Vaccine | # hatched/ | |
|---|---|---|---|
| Group | Route | # transferred | Percent hatched |
| 1 | Buffer | 17/21 | 81% |
| | in ovo | | |
| | Embryo body | | |
| 2 | Non-vaccinated | 80/101 | 79.2% |
| 3 | NDV vaccine | 18/20 | 90% |
| | in ovo | | |
| | Embryo body | | |

TABLE 10
Site of Injection using dye

| | Allantoic | Amniotic | Embryo | Yolk | |
|---|---|---|---|---|---|
| Air Cell | sac | fluid | Body | Sac | N |
| 0% | 0% | 22.0% | 78.0% | 0% | 50 |

TABLE 11
Percent hatch and NDV ELISA results of birds vaccinated in
ovo with NDV antigen, NDV antigen-Alum or an oil emulsion NDV
vaccine

| | | | | | Mean titer of |
|---|---|---|---|---|---|
| | | | Hatch | Number of | birds that |
| | | Day | of | birds | seroconverted by |
| | | 11 | Total | seroconverted/ | ELISA |
| Group | Vaccine | Boost | (%) | Total tested | (day 21 of age) |
| 1 | None | No | 96 | 0/13 | n/a |
| 2 | NDV | No | 77 | 0/14 | n/a |
| 3 | NDV | Yes | 86 | 0/14 | n/a |
| 4 | NDV-Alum | No | 88 | 8/14 | 1272 |
| 5 | NDV-OE** | No | 87 | 10/13 | 3038 |

**NDV-OE = commercial oil emulsion vaccine for NDV (LAHI)
n/a = not applicable

TABLE 12
NDV hemagglutination-inhibition results of broilers vaccinated
in ovo with NDV antigen-Alum or an oil emulsion NDV vaccine

| | | | Number of birds HI Titer ≧3/ |
|---|---|---|---|
| | | HI titer (log₂) | number of birds tested |
| Group | Treatment | Mean ± SD | (day 21 of age) |
| 2 | NDV | 1.8 ± 0.4 | 0/13 |
| 4 | NDV-Alum | 3.8 ± 1.1 | 12/14 |
| 5 | NDV-OE** | 8.9 ± 2.2 | 11/11 |

**NDV-OE = commercial oil emulsion vaccine for NDV (LAHI)

TABLE 13
Site of Injection Results

| | | Allantoic | Amniotic | Embryo | Yolk | |
|---|---|---|---|---|---|---|
| Treatment | Air Cell | sac | fluid | body | Sac | N |
| 23 G × 1.25″ | 0% | 2.1% | 16.7% | 81.3% | 0% | 48 |

TABLE 14
Percent hatch and ELISA Results of broiler chickens
vaccinated in ovo with NDV antigen or NDV-Alu,

| | | | | | Mean titer |
|---|---|---|---|---|---|
| | | | Hatch | Number of | of birds |
| | | Day | of | birds | that seroconverted |
| Group | Antigen- | 11 | Total | seroconverted/ | by ELISA |
| number | Adjuvant | Boost | (%) | Total tested | (day 21 of age) |
| 1 | None | No | 97 | 0/11 | n/a |
| 2 | NDV | No | 94* | 0/14 | n/a |
| 3 | NDV | Yes | | 1/14 | 200 |
| 4 | NDV-Alum | No | 96 | 9/14 | 411 |

*Both NDV treatments were from the same group of hatched birds; n/a = not applicable

TABLE 15
site of injection for site directed in ovo delivery of an
inactivated Newcastle disease virus oil emulsion vaccine.

| | | Site of injection | | |
|---|---|---|---|---|
| Embryo age | | Embryo | Amniotic | Allantoic |
| at injection | | body | fluid | fluid |
| Day 18.0 | | 1/34 | 33/34 | 0 |
| | % | 2.9 | 97.1 | 0 |
| Day 19 | | 57/63 | 6/63 | 0 |
| | % | 90.5 | 9.5 | 0.0 |

TABLE 16
Treatment groups and percent hatch for groups of broilers
given an oil emulsion NDV vaccine in ovo.

| Gp | | Dose | | # injected/ | Percent |
|---|---|---|---|---|---|
| # | Description¹ | (ml) | Route | # hatch | hatch |
| 1 | Non-vaccinated | NA | NA | 39/40 | 97.5 |
| 2 | NDV oil emulsion | 0.1 | Amniotic fluid in | 16/21 | 76.2 |
| | | | ovo | | |
| 3 | NDV oil emulsion | 0.1 | embryo body in | 21/21 | 100.0 |
| | | | ovo | | |
| 4 | NDV oil emulsion | 0.1 | subcutaneous | 20/20 | 100.0 |
| | | | at hatch | | |

¹NDV oil emulsion - formulated for day of age chicks, 1 dose in 0.1 ml

TABLE 17
Antibody response to NDV in broilers immunized with
an NDV oil emulsion vaccine in ovo.

| | | | Antibody titer to | | | |
|---|---|---|---|---|---|---|
| | | | NDV on day of age | | | |
| Gp # | Description¹ | Route | 13 | 21 | 26 | 35 |
| 1 | Non-vaccinated | NA | 14 | 10 | 24 | 14 |
| 2 | NDV oil emulsion | Amniotic fluid in | 5 | 8 | 23 | 34 |
| | | ovo | | | | |
| 3 | NDV oil emulsion | embryo body in | 14 | 1646 | 1645 | 2370 |
| | | ovo | | | | |
| 4 | NDV oil emulsion | subcutaneous | 16 | 1057 | 1346 | 1852 |
| | | at hatch | | | | |

¹NDV oil emulsion - formulated for day of age chicks, 1 dose in 0.1 ml

What is claimed is:

1. A method of immunizing an avian subject against Clostridium infection, comprising administering in ovo during the final quarter of incubation an effective immunizing dose of an immunogenic composition that induces an immune response against a Clostridium species, wherein the immunogenic composition is administered by in ovo injection.

2. The method of claim 1, wherein the immunogenic composition is administered to the amnion.

3. The method of claim 2, wherein the immunogenic composition is administered axially through the large end of the egg into the amniotic fluid.

4. The method of claim 1, wherein the immunogenic composition is administered directly into the embryo body.

5. The method of claim 4, wherein the immunogenic composition is administered to the embryo parenterally.

6. The method of claim 1, wherein the avian subject is a chicken.

7. The method of claim 6, wherein the immunogenic composition is administered from day 15 to day 20 of incubation.

8. The method of claim 7, wherein the immunogenic composition is administered on day 18 or 19 of incubation.

9. The method of claim 1, wherein the avian subject is a turkey.

10. The method of claim 1, wherein the immunogenic composition comprises a Clostridium perfringens toxoid.

11. The method of claim 1, wherein the immunogenic composition comprises a Clostridium perfringens bacterin.

12. The method of claim 1, wherein the immunogenic composition comprises a Clostridium perfringens toxoid and a Clostridium perfringens bacterin.

13. The method of claim 1, wherein the immunogenic composition comprises a Clostridium perfringens toxin.

14. The method of claim 13, wherein the Clostridium perfringens toxin is a Clostridium perfringens alpha toxin.

15. The method of claim 1, wherein the immunogenic composition comprises an attenuated Clostridium perfringens.

16. The method of claim 1, wherein the immunogenic composition comprises a water-in-oil-in-water emulsion.

17. The method of claim 1, wherein the immunogenic composition comprises an adjuvant.

18. The method of claim 17, wherein the adjuvant comprises an aluminum derived adjuvant, a saponin, an oil, or any combination of the foregoing.

19. The method of claim 1 further comprising administering in ovo an immune stimulant at any time during incubation.

20. The method of claim 1, further comprising administering in ovo a coccidiosis vaccine, a Marek's disease vaccine, an infectious bursal disease vaccine, a Newcastle disease vaccine, a fowl pox vaccine, or any combination of the foregoing.

21. The method of claim 20, wherein the immunogenic composition and the vaccine are administered concurrently.

22. The method of claim 21, wherein the immunogenic composition and the vaccine are administered in the same formulation.

23. The method of claim 1 further comprising administering in ovo a nutrient formulation, an enteric modulator, or a combination thereof.

24. An immunogenic composition comprising an effective immunizing dose of an attenuated Clostridium perfringens in a pharmaceutically acceptable carrier.

25. The immunogenic composition of claim 24, wherein the immunogenic composition comprises a water-in-oil-in-water emulsion.

26. The immunogenic composition of claim 24, wherein the immunogenic composition comprises an adjuvant.

27. The immunogenic composition of claim 24, wherein the immunogenic composition further comprises a coccidiosis vaccine, a Marek's disease vaccine, an infectious bursal disease vaccine, a Newcastle disease vaccine, a fowl pox vaccine, or any combination of the foregoing.

28. The immunogenic composition of claim 24, wherein the composition further comprises a nutrient formulation, an enteric modulator, or a combination thereof.

29. An immunogenic composition comprising in a pharmaceutically acceptable carrier:
(a) an effective immunizing dose of a Clostridium perfringens toxoid, a Clostridium perfringens bacterin, a C. perfringens toxin, or any combination of the foregoing; and
(b) an effective immunizing dose of a coccidiosis vaccine, a Marek's disease vaccine, an infectious bursal disease vaccine, a Newcastle disease vaccine, a fowl pox vaccine, or any combination of the foregoing.

30. An immunogenic composition comprising in a pharmaceutically acceptable carrier:
(a) an effective immunizing dose of a Clostridium perfringens toxoid, a Clostridium perfringens bacterin, a C. perfringens toxin, or any combination of the foregoing; and
(b) a water-in-oil-in-water emulsion.

31. An immunogenic composition comprising in a pharmaceutically acceptable carrier:
(a) an effective immunizing dose of a Clostridium perfringens toxoid, a Clostridium perfringens bacterin, a C. perfringens toxin, or any combination of the foregoing; and
(b) an adjuvant comprising an aluminum derived adjuvant, a saponin, an oil, or any combination of the foregoing.

32. A method of immunizing an avian subject against infection by Clostridium, comprising administering to the avian subject an effective immunizing dose of a Clostridium bacterin-toxoid composition by in ovo injection during the final quarter of incubation.

33. The method of claim 32, further comprising the step of administering a booster dose of the Clostridium bacterin-toxoid composition to the avian subject post hatch.

34. The method of claim 32, wherein the avian subject is a chicken.

35. The method of claim 32, wherein the composition further comprises an adjuvant.

36. The method of claim 32, wherein the composition comprises a Vision CD® vaccine.

37. A method of immunizing an avian subject against infection by Clostridium, comprising administering to the avian subject an effective immunizing dose of a recombinant toxin or immunogenic fragment thereof of Clostridium by in ovo injection during the final quarter of incubation.

38. The method of claim 37, further comprising the step of administering a booster dose of the recombinant toxin or immunogenic fragment thereof to the avian subject post hatch.

39. The method of claim 37, wherein the avian subject is a chicken.

40. The method of claim 37, wherein the toxin is administered with an adjuvant.

41. The method of claim 40, wherein the adjuvant is Quil A and incomplete Freund's adjuvant.

42. A method of immunizing an avian subject against necrotic enteritis, comprising administering in ovo during the final quarter of incubation an effective immunizing dose of an immunogenic composition that induces an immune response against a Clostridium species, wherein the immunogenic composition is administered by in ovo injection.


SEQUENCE LISTING (13 sequences)

SEQ ID NO: 1 - DNA, 1113 bp, Clostridium perfringens
  CDS (1)..(1110)
tgggatggaaagattgatggaacaggaactcatgctatgattgtaactcaaggggtttcaatcttagaaaatgatctgtccaaaaatgaaccagaaagtgtaagaaaaaacttagagattttaaaagagaacatgcatgagcttcaattaggttctacttatccagattatgataagaatgcatatgatctatatcaagatcatttctgggatcctgatacagataataatttctcaaaggataatagttggtatttagcttattctatacctgacacaggggaatcacaaataagaaaattttcagcattagctagatatgaatggcaaagaggaaactataaacaagctacattctatcttggagaggctatgcactattttggagatatagatactccatatcatcctgctaatgttactgccgttgatagcgcaggacatgttaagtttgaaacttttgcagaggaaagaaaagaacagtataaaataaacacagcaggttgcaaaactaatgaggatttttatgctgatatcttaaaaaacaaggattttaatgcatggtcaaaagaatatgcaagaggttttgctaaaacaggaaaatcaatatactatagtcatgctagcatgagtcatagttgggatgattgggattatgcagcaaaggtaactttagctaactctcaaaaaggaacagcaggatatatttatagattcttacacgatgtatcagagggtaatgatccatcagttggaaagaatgtaaaagaactagtagcttacatatcaactagtggtgaaaaagatgctggaacagatgactacatgtattttggaatcaaaacaaaggatggaaaaactcaagaatgggaaatggacaacccaggaaatgattttatgactggaagtaaagacacttatactttcaaattaaaagatgaaaatctaaaaattgatgatatacaaaatatgtggattagaaaaagaaaatatacagcattcccagatgcttataagccagaaaacataaagataatagcaaatggaaaagttgtagtagacaaagatataaatgagtggatttcaggaaattcaacttataatataaaataa

SEQ ID NO: 2 - protein, 370 aa, Clostridium perfringens
WDGKIDGTGTHAMIVTQGVSILENDLSKNEPESVRKNLEILKENMHELQLGSTYPDYDKNAYDLYQDHFWDPDTDNNFSKDNSWYLAYSIPDTGESQIRKFSALARYEWQRGNYKQATFYLGEAMHYFGDIDTPYHPANVTAVDSAGHVKFETFAEERKEQYKINTAGCKTNEDFYADILKNKDFNAWSKEYARGFAKTGKSIYYSHASMSHSWDDWDYAAKVTLANSQKGTAGYIYRFLHDVSEGNDPSVGKNVKELVAYISTSGEKDAGTDDYMYFGIKTKDGKTQEWEMDNPGNDFMTGSKDTYTFKLKDENLKIDDIQNMWIRKRKYTAFPDAYKPENIKIIANGKVVVDKDINEWISGNSTYNIK

SEQ ID NO: 3 - DNA, 374 bp, Clostridium perfringens
  CDS (1)..(372)
aatgatccatcagttggaaagaatgtaaaagaactagtagcttacatatcaactagtggtgaaaaagatgctggaacagatgactacatgtattttggaatcaaaacaaaggatggaaaaactcaagaatgggaaatggacaacccaggaaatgattttatgactggaagtaaagacacttatactttcaaattaaaagatgaaaatctaaaaattgatgatatacaaaatatgtggattagaaaaagaaaatatacagcattcccagatgcttataagccagaaaacataaagataatagcaaatggaaaagttgtagtagacaaagatataaatgagtggatttcaggaaattcaacttataatataaaata

SEQ ID NO: 4 - protein, 124 aa, Clostridium perfringens
NDPSVGKNVKELVAYISTSGEKDAGTDDYMYFGIKTKDGKTQEWEMDNPGNDFMTGSKDTYTFKLKDENLKIDDIQNMWIRKRKYTAFPDAYKPENIKIIANGKVVVDKDINEWISGNSTYNIK

SEQ ID NO: 5 - DNA, 834 bp, Clostridium perfringens
  CDS (1)..(834)
tgggatggaaagattgatggaacaggaactcatgctatgattgtaactcaaggggtttcaatcttagaaaatgatctgtccaaaaatgaaccagaaagtgtaagaaaaaacttagagattttaaaagagaacatgcatgagcttcaattaggttctacttatccagattatgataagaatgcatatgatctatatcaagatcatttctgggatcctgatacagataataatttctcaaaggataatagttggtatttagcttattctatacctgacacaggggaatcacaaataagaaaattttcagcattagctagatatgaatggcaaagaggaaactataaacaagctacattctatcttggagaggctatgcactattttggagatatagatactccatatcatcctgctaatgttactgccgttgatagcgcaggacatgttaagtttgaaacttttgcagaggaaagaaaagaacagtataaaataaacacagcaggttgcaaaactaatgaggatttttatgctgatatcttaaaaaacaaggattttaatgcatggtcaaaagaatatgcaagaggttttgctaaaacaggaaaatcaatatactatagtcatgctagcatgagtcatagttgggatgattgggattatgcagcaaaggtaactttagctaactctcaaaaaggaacagcaggatatatttatagattcttacacgatgtatcagagggtaatgatccatcagttggaaagaatgtaaaagaactagtagcttacatatcaactagtggtgaaaaagatgctggaacagatgactacatgtatttt

SEQ ID NO: 6 - protein, 278 aa, Clostridium perfringens
WDGKIDGTGTHAMIVTQGVSILENDLSKNEPESVRKNLEILKENMHELQLGSTYPDYDKNAYDLYQDHFWDPDTDNNFSKDNSWYLAYSIPDTGESQIRKFSALARYEWQRGNYKQATFYLGEAMHYFGDIDTPYHPANVTAVDSAGHVKFETFAEERKEQYKINTAGCKTNEDFYADILKNKDFNAWSKEYARGFAKTGKSIYYSHASMSHSWDDWDYAAKVTLANSQKGTAGYIYRFLHDVSEGNDPSVGKNVKELVAYISTSGEKDAGTDDYMYF

SEQ ID NO: 7 - DNA, 120 bp, Clostridium perfringens
  CDS (1)..(120)
tacatatcaactagtggtgaaaaagatgctggaacagatgactacatgtattttggaatcaaaacaaaggatggaaaaactcaagaatgggaaatggacaacccaggaaatgattttatg

SEQ ID NO: 8 - protein, 40 aa, Clostridium perfringens
YISTSGEKDAGTDDYMYFGIKTKDGKTQEWEMDNPGNDFM

SEQ ID NO: 9 - DNA, 1197 bp, Clostridium perfringens
  CDS (1)..(1194)
atgaaaagaaagatttgtaaggcgcttatttgtgctacgctagcaactagcctatgggctggggcatcaactaaagtctacgcttgggatggaaagattgatggaacaggaactcatgctatgattgtaactcaaggggtttcaatcttagaaaatgatctgtccaaaaatgaaccagaaagtgtaagaaaaaacttagagattttaaaagagaacatgcatgagcttcaattaggttctacttatccagattatgataagaatgcatatgatctatatcaagatcatttctgggatcctgatacagataataatttctcaaaggataatagttggtatttagcttattctatacctgacacaggggaatcacaaataagaaaattttcagcattagctagatatgaatggcaaagaggaaactataaacaagctacattctatcttggagaggctatgcactattttggagatatagatactccatatcatcctgctaatgttactgccgttgatagcgcaggacatgttaagtttgaaacttttgcagaggaaagaaaagaacagtataaaataaacacagcaggttgcaaaactaatgaggatttttatgctgatatcttaaaaaacaaggattttaatgcatggtcaaaagaatatgcaagaggttttgctaaaacaggaaaatcaatatactatagtcatgctagcatgagtcatagttgggatgattgggattatgcagcaaaggtaactttagctaactctcaaaaaggaacagcaggatatatttatagattcttacacgatgtatcagagggtaatgatccatcagttggaaagaatgtaaaagaactagtagcttacatatcaactagtggtgaaaaagatgctggaacagatgactacatgtattttggaatcaaaacaaaggatggaaaaactcaagaatgggaaatggacaacccaggaaatgattttatgactggaagtaaagacacttatactttcaaattaaaagatgaaaatctaaaaattgatgatatacaaaatatgtggattagaaaaagaaaatatacagcattcccagatgcttataagccagaaaacataaagataatagcaaatggaaaagttgtagtagacaaagatataaatgagtggatttcaggaaattcaacttataatataaaataa

SEQ ID NO: 10 - protein, 398 aa, Clostridium perfringens
MKRKICKALICATLATSLWAGASTKVYAWDGKIDGTGTHAMIVTQGVSILENDLSKNEPESVRKNLEILKENMHELQLGSTYPDYDKNAYDLYQDHFWDPDTDNNFSKDNSWYLAYSIPDTGESQIRKFSALARYEWQRGNYKQATFYLGEAMHYFGDIDTPYHPANVTAVDSAGHVKFETFAEERKEQYKINTAGCKTNEDFYADILKNKDFNAWSKEYARGFAKTGKSIYYSHASMSHSWDDWDYAAKVTLANSQKGTAGYIYRFLHDVSEGNDPSVGKNVKELVAYISTSGEKDAGTDDYMYFGIKTKDGKTQEWEMDNPGNDFMTGSKDTYTFKLKDENLKIDDIQNMWIRKRKYTAFPDAYKPENIKIIANGKVVVDKDINEWISGNSTYNIK

SEQ ID NO: 11 - protein, 336 aa, Clostridium perfringens
MKKKFISLVIVSSLLNGCLLSPRLVYANDIGKTTTITRNKTSDGYTIITQNDKWIISYQSVDSSSKNEDGFTASIDARFIDDKYSSEMTTLINLTGFMSSKKEDVIKKYNLHDNTNSTAINFPVRYSISILNESINENVKIVDSIPKNTISQKTVSNTMGYKIGGSIEIEENKPKASIESEYAESSTIEYVQPDFSTIQTDHSTSKASWDTKFTETTRGNYNLKSNNPVYGNEMFMYGRYTNVPATENIIPDYQMSKLITGGLNPNMSVVLTAPNGTEESIIKVKMERERNCYYLNWNGANWVGQVYSRLAFDTPNVDSHIFTFKINWLTHKVTAI

SEQ ID NO: 12 - protein, 328 aa, Clostridium perfringens
MKKNLVKSLAIASAVISIYSIVNIVSPTNVIAKEISNTVSNEMSKKASYDNVDTLIEKGRYNTKYNYLKRMEKYYPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITHNVPSQDILVPANTTVEVIAYLKKVNVKGNVKLVGQVSGSEWGEIPSYLAFPRDGYKFSLSDTVNKSDLNEDGTININGKGNYSAVMGDELIVKVRNLNTNNVQEYVIPVDKKEKSNDSNIVKYRSLYIKAPGIK

SEQ ID NO: 13 - protein, 283 aa, Clostridium perfringens
KASYDNVDTLIEKGRYNTKYNYLKRMEKYYPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITHNVPSQDILVPANTTVEVIAYLKKVNVKGNVKLVGQVSGSEWGEIPSYLAFPRDGYKFSLSDTVNKSDLNEDGTININGKGNYSAVMGDELIVKVRNLNTNNVQEYVIPVDKKEKSNDSNIVKYRSLYIKAPGIK